(19) United States
(12) Patent Application Publication
(10) Pub. No.: US 2023/0393064 A1
(43) Pub. Date: Dec. 7, 2023

(54) METHOD AND APPARATUS FOR MEASUREMENT OF AN ANALYTE

(71) Applicant: University of Durham, Durham (GB)

(72) Inventors: David CARTY, Durham (GB); Eckart WREDE, Durham (GB); Nils Hendrik NAHLER, Midlothian (GB)

(21) Appl. No.: 18/044,759

(22) PCT Filed: Sep. 10, 2021
(86) PCT No.: PCT/GB2021/052345

§ 371 Date: Mar. 9, 2023

(30) Foreign Application Priority Data
Sep. 10, 2020 (GB) 2014270.9

(51) Int. Cl. G01N 21/64; A61B 5/097; G01N 33/497; G01N 21/39; G01J 3/44; G01N 21/05

(52) U.S. Cl. CPC G01N 21/6402; A61B 5/097; G01N 33/497; G01N 21/39; G01J 3/4406; G01N 21/05; G01N 2021/391

(57) ABSTRACT

The invention relates to a method for measurement and monitoring of analytes, for example ketones, particularly but not exclusively acetone in human or other mammalian breath. The invention also relates to apparatus for use in performance of the method. The present invention uses a spectroscopic technique known as CELIF which is a direct combination of the well-established and powerful laser-spectroscopic techniques cavity ring-down spectroscopy and laser-induced fluorescence. The method utilises a flow body to control a flow of sample gas through a laser beam.

This invention relates to a method for measurement and monitoring of ketones, particularly but not exclusively acetone in human or other mammalian breath. The invention also relates to apparatus for use in performance of the method.

Diabetes mellitus is a group of metabolic diseases where defects in insulin action and/or secretion results in harmfully high blood glucose (BG) levels. Effective diabetes management can greatly reduce the risk of complications. However, when poorly managed the disease can cause serious complications such as cardiovascular disease, kidney disease, blindness, amputation, diabetic ketoacidosis (DKA), depression, neuropathy and sexual dysfunction. Around 366 million people worldwide had diabetes in 2011. In the UK, 2.9 million people have been diagnosed with diabetes and it has been estimated that a further 850,000 are sufferers of the condition but have yet to be diagnosed. Including children, 15% have the non-preventable type I form (T1D), characterised by a lack of insulin production and the remaining fraction have the preventable type II form (T2D), which is linked with obesity and lifestyle. Children with diabetes have the worst rates of very high-risk glucose control. In 2009/10, 9% of children with diabetes experienced at least one episode of acute DKA, a fast-developing and life-threatening emergency. DKA is caused by the build-up of body ketones, which are produced when the body burns fat instead of glucose. Some people with undiagnosed T1D do not receive a diagnosis until they are seriously ill with DKA.

DKA kills more than ten young people with T1D each year in the UK. The severity of DKA at diagnosis is inversely related to neurocognitive outcome leading to substantial subsequent health costs. Timely diagnosis is essential, yet primary and secondary healthcare teams frequently miss T1D/DKA diagnosis. The proportion of cases presenting DKA has not changed for many years despite intervention programmes, which reflects the difficulty in obtaining blood or urine samples. Hospital-based insulin treatment of DKA (in both children and in adults) uses measurement of blood pH and measurement of the blood ketone/β-hydroxybutyrate to monitor response, but this is often problematic and inadequate—sampling can be difficult and the results of ketone measurement can be inaccurate, samples may need to be sent to a different location for analysis leading to untimely reporting of results and blood pH may remain low due to emerging hypochloraemia caused by saline treatment thus masking the response to treatment being measured.

Ketoacidosis can also be caused by starvation as a result of dieting or illness. The symptoms of ketoacidosis can appear in children, even in those that do not have T1D, and are often mistaken for food poisoning, which can lead to delays in appropriate treatment. Pregnant women with T1D during labour are susceptible to DKA brought on by the medical stress they are in. People with severe epilepsy can be treated with a ketogenic diet, which is effective only if the person remains ketotic thus making the diet extremely difficult to deliver and maintain safely. Ketosis can be induced intentionally to promote weight loss or to enhance sports performance in particular in endurance athletes. Ketosis induced by a ketogenic diet or by fasting can lead to breath acetone concentrations up to 200 ppm.

A non-invasive method for diagnosis and monitoring of T1D/DKA as an alternative to blood analysis is the measurement of the concentration of the diabetes biomarker acetone in exhaled breath (Ref: T. P. J. Blaikie, et al., J. Breath Res., 8 (2014) 046010). Sensitive quantification of the very low concentrations of breath acetone is difficult, particularly acetone over the entire range experienced in DKA patients (0.5 ppm-2,000 ppm). The presence of other trace gases in breath, including isoprene, acetoacetate and β-hydroxybutyrate, which are also associated with diabetes, as well as high concentrations of water, carbon dioxide, nitrogen and oxygen, means that high selectivity is required. For an acetone measurement device to be effective in a point-of-care (POC) setting, it should be portable outside a laboratory environment, affordable and offer a real-time, on-line measurement reported directly and continuously from the patient. Such a device would thus remove any uncertainty a clinician may have and can ensure that treatment decisions are applied with confidence.

A large number of techniques for laser spectroscopic breath analysis have been used. These are reviewed in V. Ruzsinyi and M. P. Kalapos, J. Breath Res., 11 (2017) 024002: Breath acetone as a potential marker in clinical practice. In order to compare techniques, individual techniques must weigh up against five criteria, all of which would be ideally satisfied: sensitivity, i.e. can an acceptably small change in analyte concentration be detected; selectivity—does the detection measure the target analyte without significant interference from other chemical species; POC—is the device portable enough to reside next to the patient; real time—can the correct measurement be made and results given back without an unacceptable delay; and online—can the sample go directly from the patient into the device. Table 1 summarises the criteria that can be met by the techniques that have been used for the measurement of acetone in breath.

TABLE 1
Summary of whether a particular technique meets criteria to an
effective point-of- care device.

| Technique | Sensitivity | Selectivity | POC | Real time | Online |
|---|---|---|---|---|---|
| SPME-GC-MS | ✓ | ✓ | X | X | X |
| PTR-MS/SIFT-MS | ✓ | ✓ | X | ✓ | ✓ |
| Electrochemical | ✓ | X | ✓ | X | X |
| Si: WO₃ sensor | X | X | ✓ | ✓ | ✓ |
| CEAS | X | ✓ | ✓ | X | X |
| CRDS | X | ✓ | ✓ | ✓ | ✓ |
| ICOS | ✓ | ✓ | ✓ | X | ✓ |
| CELIF | ✓ | ✓ | ✓ | ✓ | ✓ |

SPME-GC-MS, solid-phase micro-extraction mass-spectrometry; PTR-MS, proton-transfer-reaction mass spectrometry; SIFT-MS, selected-ion flow-tube mass spectrometry; Si: WO₃ sensor, chemo-resistive sensor using Si-doped tungsten trioxide nanoparticles; CEAS, cavity-enhanced absorption spectroscopy; CRDS, cavity ring-down spectroscopy; ICOS, integrated cavity-output spectroscopy and CELIF, cavity-enhanced laser-induced fluorescence.

Gas chromatography (GC) has been used in breath analysis. This is used to isolate a particular breath analyte, such as acetone, where it is then detected—typically by mass spectrometry (MS). While this technique easily reaches the required sensitivity and selectivity, the instrumentation is prohibitively expensive for POC use. Real-time, on-line analysis is impossible because breath samples must be pre-concentrated using a technique such as solid-phase micro-extraction (SPME). Other MS techniques such as proton-transfer-reaction MS (PTR-MS) and selected-ion flow-tube MS (SIFT-MS) are better, but the instrumentation is not suitable for POC use on the grounds of size and cost. Electrochemical devices, such as a multi-wall carbon-nanotube/SnO₂ sensor, can be sensitive enough and may be good for POC use by virtue of being inexpensive and portable, but they do not meet any of the other criteria. The recent Si:WO₃ chemo-resistive sensor [M. Righettoni et al., Monitoring breath markers under controlled conditions, J. Breath Res., 2015, 9, 047101], shows good correlation with PTR-TOF-MS over a limited dynamic range and requires specific breathing protocols. The laser-spectroscopy-based techniques cavity-enhanced absorption spectroscopy (CEAS), cavity ring-down spectroscopy (CRDS) and integrated cavity-output spectroscopy (ICOS) that have been used in acetone breath analysis have proved to be more promising. CEAS lacks sensitivity and the measurement must be done offline and not in real time using a sample bag that is taken to the device. CRDS only lacks sensitivity and ICOS only lacks the ability to obtain a measurement fast enough for real time results. Cavity-enhanced laser-induced fluorescence (CELIF) meets all desired criteria.

The present invention uses CELIF which is a direct combination of the well-established and powerful laser-spectroscopic techniques cavity ring-down spectroscopy and laser-induced fluorescence.

Cavity ring-down spectroscopy (CRDS) measures the absorption of light by the sample. CRDS is implemented by injecting a laser pulse into a stable optical cavity consisting of two highly reflective mirrors. When a laser pulse is incident on the back side of the first mirror, the majority of the pulse is reflected away from the cavity. However, a small portion of the pulse, where the size of the portion is dependent on the finite reflectivity of the mirror, is injected into the optical cavity. When this injected pulse encounters the second highly reflective mirror, the majority of the remaining pulse is reflected back into the cavity while a minor fraction is transmitted and leaves the cavity. The laser pulse reflects back and forth between the mirror surfaces transmitting a small percentage of its intensity with each mirror encounter. The transmitted intensity usually follows an exponential decay and is monitored by a photodetector located behind the second mirror. With an “empty” cavity, i.e. without an absorbing species in the cavity (vacuum, sample without analyte or laser detuned from any absorption line), the time constant of the decay, called the ring-down time (RDT), is a measure of the reflectivity of the mirrors. Following introduction of an absorbing analyte species or tuning the laser onto an absorption line of a species in the cavity, the absorption increases leading to a faster decay and a shorter ring-down time. The decrease in the ring-down time depends on the absorption strength (absorption cross section) of the species, the particular wavelength and also the concentration of the species in the cavity. The concentration for the analyte is determined from the reduction in the ring-down time compared to the “empty” cavity and literature values for the absorption strength of the analyte. CRDS is able to determine absolute concentrations.

Laser-induced fluorescence (LIF) is an indirect detection technique. When an analyte molecule absorbs light it is excited into a higher energy state. This excited molecule can release its energy by emitting light (fluorescence) that is detected by a photodetector at an angle to the laser beam. However, other processes can prevent fluorescent light being emitted, hence, fluorescence by the molecule is a requirement. The amount of light detected depends on the intensity and wavelength of the incident laser light, the analyte concentration, the absorption strength of the analyte, the fraction of light emitted with respect to the light absorbed (fluorescence quantum yield of the analyte) and the efficiency of the LIF detection system. In general, only relative concentrations (e.g. when comparing two samples under otherwise identical conditions) can be determined via LIF. In order to determine the absolute analyte concentrations from the intensity of the detected fluorescence a complex and protracted calibration of the detection system is required.

Cavity-enhanced laser-induced fluorescence (CELIF) combines both the LIF and CRDS techniques in a single setup, using the same laser beam and detecting the same analyte molecules. The LIF aspect is used to detect the analyte. The optical cavity of the CRDS aspect very effectively eliminates stray light—a significant problem in traditional LIF implementations. The CRDS aspect is also used to correct the LIF for fluctuations of the intensity of the laser pulses (normalisation) and to provide the absolute calibration of the LIF detection system based on neat acetone/N₂ calibration samples. At high concentrations, the reduction of the ring-down time (via CRDS measurement) yields an absolute measure of the analyte concentration which directly calibrates the intensity of the simultaneously measured fluorescence by the LIF detection system. The limit of detection (LOD) of the CRDS technique is reached when the ring-down time becomes indistinguishable from the ring-down time of a sample without analyte. Generally, the LOD of LIF is much lower compared to CRDS, and due to the cross calibration, much lower absolute concentration can be detected via CELIF. The LOD of CELIF is defined by the LIF background signal (without analyte) due to remaining stray and scattered light and the electronic noise of the detection system. CELIF is able to determine absolute concentrations well below the LOD of CRDS. CELIF is also ideally suited to detect very high acetone concentrations (>100 ppm) when CRDS becomes unsuitable.

WO2017/021424 discloses apparatus for quantifying an analyte in a gas or liquid phase sample comprising a CELIF spectrophotometer.

According to a first aspect of the present invention there is provided an apparatus for quantifying an analyte in a gas or liquid phase sample comprising:
a light source with an emission spectrum that overlaps with an absorption of the analyte, a pair of reflective mirrors located on an optical axis to form an optical cavity, the cavity having a sample inlet and a sample outlet;
a fluorescence detector located at a location not on the cavity axis and arranged to provide a first signal in response to fluorescence within the cavity;
a photon detector located axially external to the cavity and arranged to provide a second signal;
wherein the apparatus including the light source, the cavity and the axial photon detector comprises a cavity-enhanced absorption spectrometer;
wherein the apparatus including the fluorescence detector is configured to comprise a cavity-enhanced laser-induced fluorescence (CELIF) spectrophotometer;
means for supplying an analyte-free gas sample or an analyte-containing gas sample to the cavity through the inlet;
a processor adapted to receive a first signal from the fluorescence detector and a second signal from the axial photon detector, and further adapted to provide a measurement of the analyte concentration in the sample:
wherein a flow body is located between the sample inlet and sample outlet, the flow body comprising a chamber extending along a direction of sample flow between the sample inlet and the sample outlet, the flow body further comprising a light source inlet (such as a laser beam inlet) and a light source outlet (such as a laser beam outlet) arranged to provide a path for a light source (such as a laser beam) through the chamber, the path extending transversely of the direction of sample flow;
the flow body further comprising an aperture communicating with the fluorescence photon detector in a direction transverse to the path of the light source (optionally the laser beam).

The present invention enables quantitative measurement of gaseous acetone by cavity-enhanced laser-induced fluorescence (CELIF) to be made in real time. This enables acetone concentration that is varying in time to be accurately followed. The use of a flow body as defined above enables such real time measurement.

In embodiments, the light source may be a laser.

In embodiments, the light source may be a sub-nanosecond-pulsed Nd:YAG laser, optionally operating with a wavelength output of 266 nm at a repetition rate of 15 kHz.

In embodiments the flow body further comprises one or more side flow channels.

A further important feature is that, when calibrating the CELIF device used in this invention, the calibration gas which fills the cavity must be kept flowing. This avoids “bleaching” of the sample, where the absorption of photons effectively removes analyte molecules over the timescale of the measurement artificially reducing the reading, as this can lead to an inaccurate measurement of the analyte concentration.

The present invention arises from the discovery that in order to measure a time-varying gas concentration in a sample, the spatial extent of the probe laser must be minimised such that the gas passes quickly through the probe laser. This is because in a flowing gas, a time-varying concentration is equivalent to a spatially-varying concentration. Sufficient resolution of a time-varying concentration is, therefore, best achieved by flowing the gas along an axis perpendicular to the optical cavity axis (transverse flow). Devices in which the volume being measured is large include cavity ring-down spectroscopy (CRDS) devices and SIFT (and PTR) mass spectrometry (MS) devices. In SIFT (and PTR) MS these large volumes can be achieved by diluting the sample with air or nitrogen, generally without sacrificing the sensitivity of the instrument, provided that the dilution ratio is carefully monitored. In CRDS, dilution is typically not an option as this will lead to concentrations which are lower than the limit of detection.

Further advantage of flow of the sample transverse to the fluorescence detector is that it avoids bleaching of the sample. Bleaching may occur when a molecule absorbs a photon during measurement but remains in the volume being measured for a period that is longer than the intervals between measurements, thereby effectively lowering the sample concentration. The flow speed must be fast enough to remove a molecule from the measurement area, which is easier with transverse flow of the sample. The invention finds particular application in detection of acetone in time-resolved profiles of exhaled breath. The invention finds application in medical devices to inform the treatment of patients with diabetic ketoacidosis and to control the induced ketosis of patients with epilepsy. A further application is in sports and exercise physiology where the measurement of breath ketones allows control of an athlete's ketogenic diet or intended state of ketosis for performance enhancement.

Real time measurement of acetone concentration of exhaled breath is enabled by the flow body of the apparatus of the present invention. In particular, the apparatus of the present invention allows for a rapid response to changes in the acetone concentration. A rapid response to the acetone concentration may be achieved by having an apparatus where the CELIF signal rises and reaches the maximum acetone concentration (for example the upper limit acetone concentration tolerated by the apparatus or the maximum acetone concentration that will trigger an alert of a high level of acetone) in less than 1 s, then fall and return to the baseline level before the next breath arrives.

The length of time taken for the signal to rise from 10% to 90% of the maximum acetone concentration, with respect to the background concentration, is referred to as the rise time. The length of time taken for the signal to fall back to the baseline level is referred to as the fall time.

The flow rate through the flow body may be controlled by a mass flow controller. As such, in embodiments the apparatus further includes a mass flow controller. The flow rate of the sample may be referred to as the sample flow rate. The sample flow rate may be controlled by the rate of exhalation of a patient who is being monitored by being attached to the apparatus. However, the sample flow rate can also be controlled by the mass flow controller. The sample flow rate may be greater than 0.1 slpm. In embodiments the sample flow rate may be greater than or equal to 0.5 slpm.

The chamber, that is the internal dimension of the flow body, may have an internal dimension perpendicular to the direction of sample flow (which may be considered as a width), the dimension increasing from a minimum value at the sample inlet to a maximum value in the vicinity of the light source path (optionally the laser beam path) and decreasing to a value smaller than the maximum value at the sample outlet.

The dimensions of the chamber increase or decrease smoothly. The chamber increasing or decreasing smoothly provides that no corners or sharp edges are presented to the sample flow. Sharp edges or discontinuities within the wall of the chamber can induce eddies in the gas flow that can recirculate gas in the detection region thereby increasing rise and fall times. Thus, certain embodiments of the present invention is concerned with addressing the problem of reducing rise and fall times by avoiding eddies and recirculation in the sample flow.

A maximum dimension of the chamber may be parallel to the laser beam path. In an embodiment, the maximum dimension may be located along the laser beam path. In an alternative embodiment the maximum dimension is not located along the laser beam path.

In an embodiment, the direction of sample flow, the laser beam path and the fluorescence detector aperture are perpendicular or orthogonal and intersect at a single point. Accordingly, at the single point there are three axes or directions that are converging. In such an arrangement, the construction of the apparatus is simplified. The function of the apparatus is most efficient if the three directions are all perpendicular to each other but alternative configurations, for example two perpendicular axes may be employed.

In order to detect fluorescence from the sample, the field of view of the fluorescence detection system needs to include the excitation volume defined as the intersection volume of the sample flow and laser beam.

The surface of the chamber may have a smooth profile configured to minimise turbulent flow of the sample gas, or formation of eddies, during passage through the chamber.

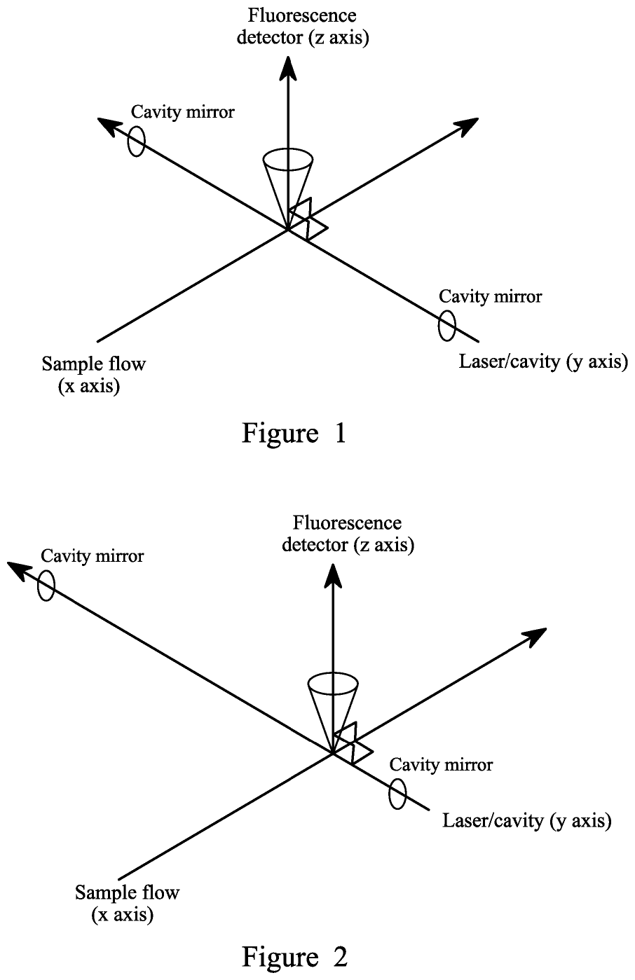

The width of a cross section of the chamber taken parallel to the sample flow (in the x-z plane and/or the x-y plane, referring to FIG. 1) may increase gradually from the inlet to a maximum value in the vicinity of the laser beam path (for example just before the laser beam path) and may decrease towards the outlet, so that the velocity of the sample gas decreases as it flows towards the vicinity of the laser beam and increases from the vicinity of the laser beam to the outlet.

In embodiments, the apparatus further comprises a breath collector connected to an inlet of apparatus as claimed in any preceding claim.

According to a second aspect of the present invention, a method of quantifying an analyte in a gas phase sample comprises the steps of:
providing apparatus in accordance with the first aspect of the invention;
introducing an analyte-free gas sample into the chamber obtaining a background signal; introducing a flowing reference sample containing a quantity of the analyte into the chamber and obtaining a reference signal;
processing the background signal and reference signal to obtain an absolute absorption coefficient of the reference sample and to provide a calibration signal of the CELIF spectrophotometer;
introducing a specimen gas sample containing the analyte into the chamber obtaining a specimen signal;
processing the specimen signal and the calibration signal to obtain a measurement of the absolute concentration of the analyte in the specimen sample;
wherein the measurement is made while the specimen sample is flowing through the chamber.

In embodiments, the specimen sample is a breath sample, optionally wherein the measurement is made while the breath sample is flowing through the flow body whilst a patient is breathing into the apparatus under normal breathing conditions.

In embodiments the analyte concentration is measured repeatedly over time intervals of 100 ns to 5 ms. In embodiments an average value for the analyte concentration is measured over a period from 1.0 ms to 0.03 s.

In an embodiment, the method includes the step of determining a value for a background signal; and
subtracting the background signal from the specimen signal to provide a corrected specimen signal.

The background signal may comprise: for background that depends on the light (laser) intensity, e.g. stray light or fluorescence of the mirrors, is laser-independent background, e.g. PMT dark counts and leaked ambient light, gamma cosmic rays, black body radiation from heated appliances and thermionic radiation

The total detected fluorescence, S^{LIF}, will include background contributions and will be calculated as set out:

Sₜₒₜ^{LIF}=αKS^{CRD}+βS^{CRD}+γ

in which βS^{CRD} and γ account for the laser-dependent and laser-independent background, respectively.

In a further aspect of the present invention there is provided a method of quantifying an analyte in a patients breath comprising the steps of:
providing apparatus in accordance with the first aspect of the invention;
introducing an analyte-free gas sample into the chamber obtaining a background signal;
introducing a flowing reference sample containing a quantity of the analyte into the chamber and obtaining a reference signal;
processing the background signal and reference signal to obtain an absolute absorption coefficient of the reference sample and to provide a calibration signal of the CELIF spectrophotometer;
introducing a specimen gas sample containing the analyte into the chamber obtaining a specimen signal, wherein the specimen gas sample is the patients breath;
processing the specimen signal and the calibration signal to obtain a measurement of the absolute concentration of the analyte in the specimen sample;
wherein the measurement is made while the breath sample is flowing through the chamber.

In embodiments, the method further comprises a step of cleaning the chamber by passing a gas through the chamber. In embodiments, the chamber is cleaned every 2 minutes, optionally every 1 minute. In embodiments, the time between cleaning is dependent on the concentration of analyte in the gas sample. Accordingly, in embodiments the method further comprises the step of cleaning the chamber by passing a gas through the chamber every 2 minutes where the gas sample has an analyte concentration of less than 30 ppm and every 1 minute where the gas sample has an analyte concentration of greater than 30 ppm (optionally with an upper limit of the upper detection limit of the apparatus, e.g. 2000 ppm).

In embodiments, the method further comprises a step of cleaning the chamber by passing a gas through the chamber. In embodiments the chamber is cleaned by passing a gas along the sample path or along the laser beam path, alternatively a gas may be drawn in through the same beam path (for example at both mirrors) and pumped out in the centre along the sample path. In embodiments, the chamber is cleaned every 2 minutes when breath is sampled continuously, optionally every 1 minute when the analyte concentration is greater than 30 ppm. In embodiments, the time between cleaning is dependent on the concentration of analyte in the gas sample. For end-tidal breath sampling, cleaning is required after each patient. Accordingly, in embodiments the method further comprises the step of cleaning the chamber by passing a gas through the chamber at an increased flow rate of greater than 2 slpm to reduce the cleaning time. In the embodiment, the cleaning cycle for analyte concentrations below 30 ppm comprises air purging for 2 minutes along the sample path, followed by 2 minutes along the laser path and finished by 1 minute along the sample path. For analyte concentrations greater than 30 ppm the air purging times are doubled.

In embodiments the cleaning step happens between patients or if there are more than 30 min between measurements of the same patient. The duration of the purging cycle along the sample path and along the laser path is correlated to the sampled analyte concentrations with a 2 min cycle at a 30 ppm analyte concentration.

In a further aspect of the present invention, there is provided a method of detecting or monitoring Type 1 diabetes in a patient comprising the step of measuring acetone in exhaled breath using the method of another aspect of the present invention.

In a further aspect of the present invention, there is provided a method of detecting ketone acidosis in a patient comprising the step of:
measuring acetone in exhaled breath using the apparatus of the present invention.

The present invention is now discussed in more detail with reference to the Figures.

FIG. 1 shows the geometry of a preferred embodiment where the laser/cavity axis intersects the sample flow at right angles. The fluorescence is detected along a third axis perpendicular to both. None of the housings including the flow body are drawn.

FIG. 2 shows the intersection of the laser by the sample flow can be at any point along the laser beam within the cavity. The axis of the fluorescence detection optics intersect at the same point.

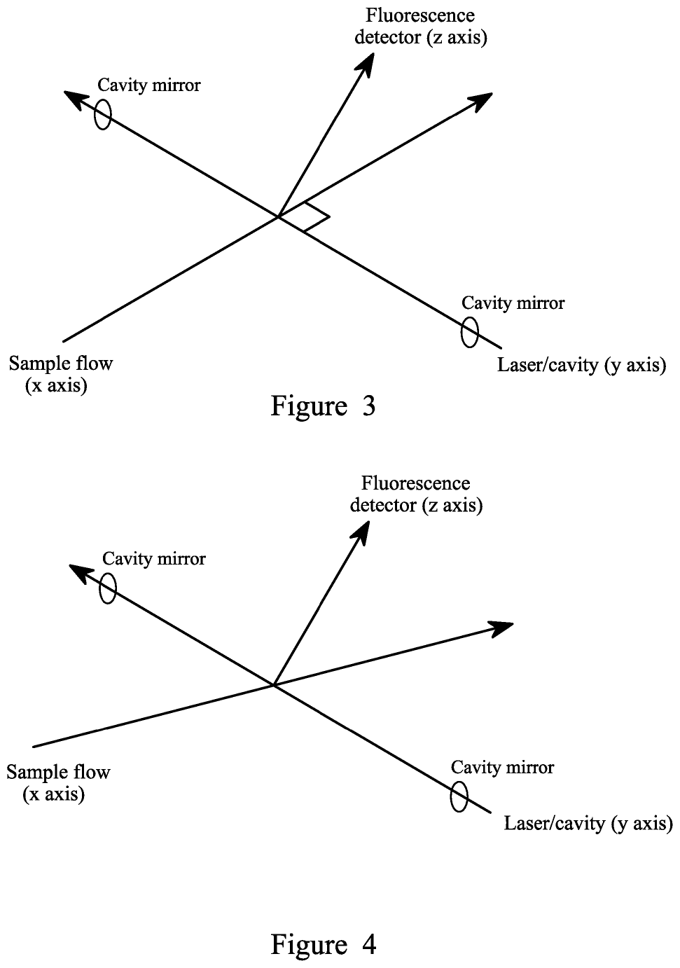

FIGS. 3 and 4 show that the axis of the fluorescence detection optics/system does not need to be at right angles to either the laser/cavity or sample flow axes (FIG. 3) and that the sample flow does not need to intersect the laser/cavity axis at right angles (FIG. 4).

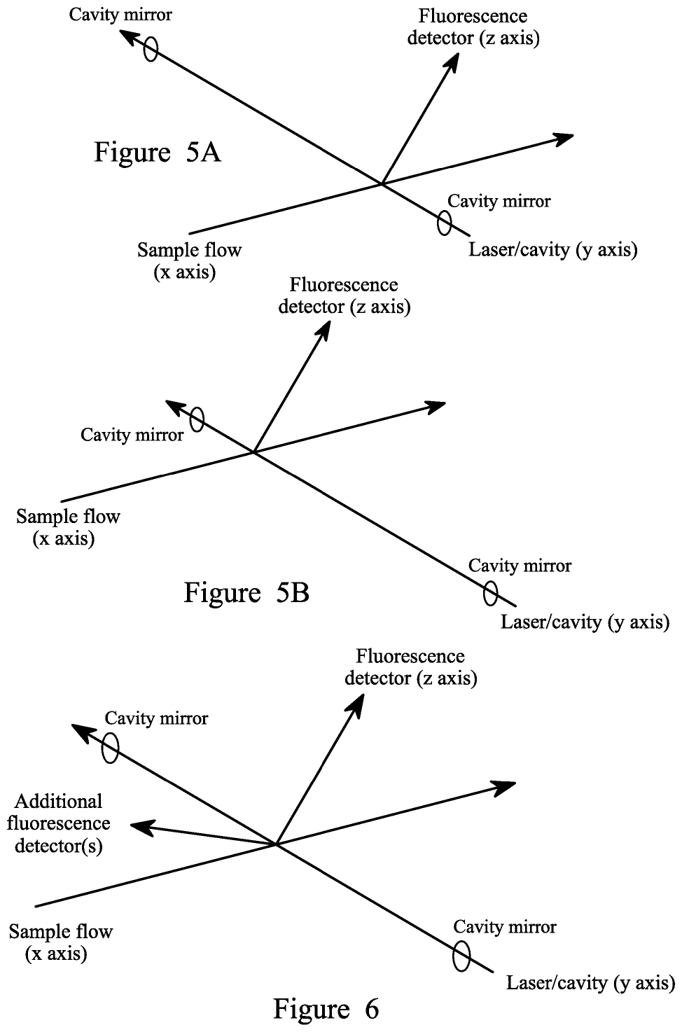

FIGS. 5A and 5B show that the excitation volume can be anywhere along the laser beam within the cavity.

FIG. 6 shows an embodiment with a fluorescence detection system consisting of multiple detectors and fluorescence optics located along different axes, a detector array arranged around the excitation volume, or an integration sphere (with suitable holes/openings for the laser beam and the sample flow).

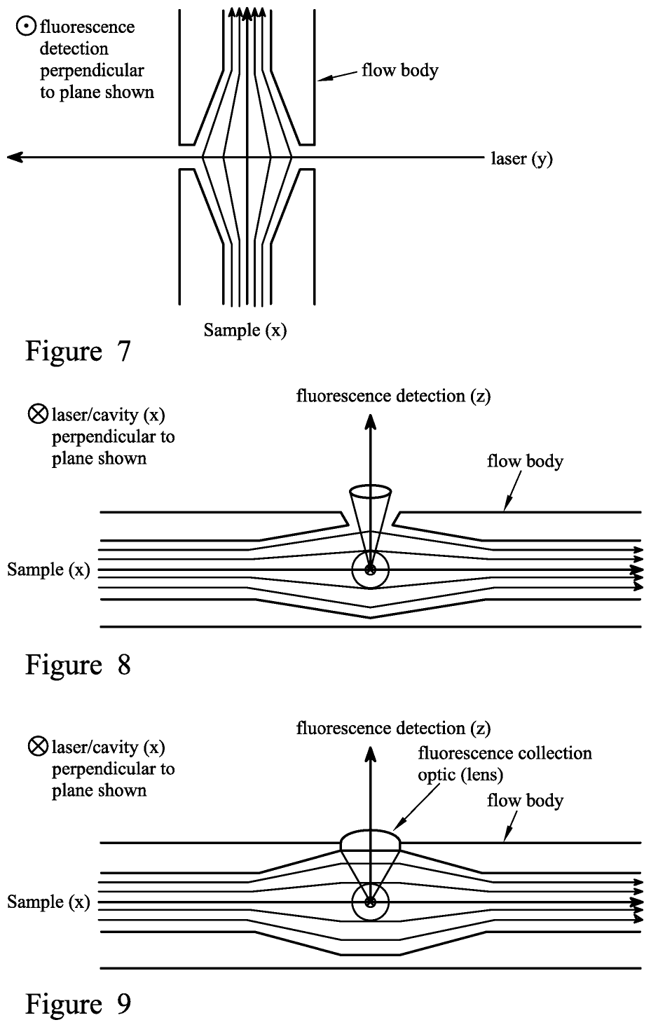

FIG. 7 shows a schematic sample flow in the plane spanned by the sample flow and laser axes, showing how the chamber may expand in the dimension of the laser axis.

FIG. 8 shows a schematic view of sample flow in the plane spanned by the sample flow and the fluorescence detection. The sample flow may expand in the dimension of the fluorescence detection axis.

FIG. 9 shows a schematic view of sample flow in the plane spanned by the sample flow and the fluorescence detection. The first lens of the fluorescence detection optics may form the internal boundary of the flow body.

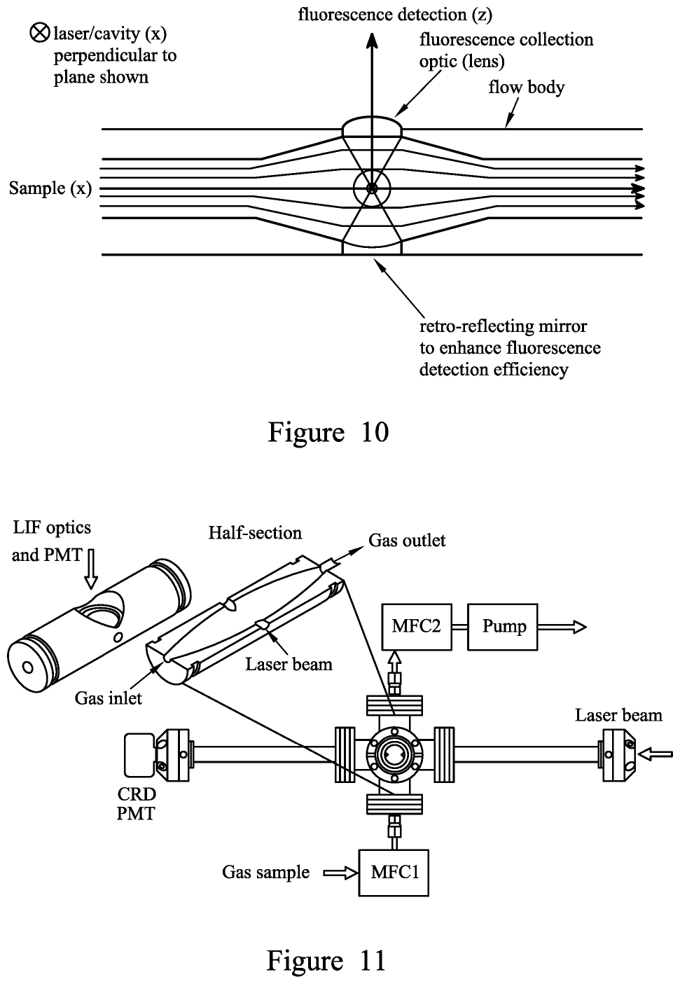

FIG. 10 shows a schematic view of sample flow in the plane spanned by the sample flow and the fluorescence detection. A retro-reflecting concave mirror is placed opposite the fluorescence detection optics to increase the amount of fluorescence light that can be detected.

FIG. 11 shows a half-section view of the flow body when viewed from the top (the x-y plane) and the six way cross configuration of the apparatus for quantifying an analyte in a gas or liquid phase.

FIG. 12 shows signals of different standard acetone-nitrogen gas mixtures were measured once with longitudinal flow and once with transverse flow, with a flow rate of 0.5 slpm in each flow direction.

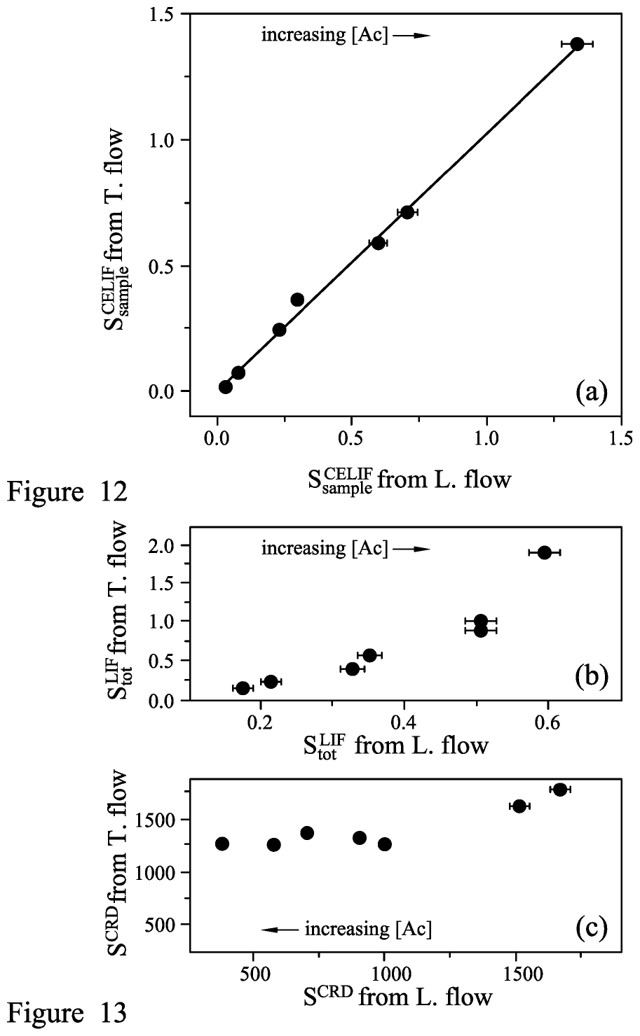

FIG. 13 shows a comparison of the LIF and CRD signals between transverse and longitudinal flow of the same gas samples

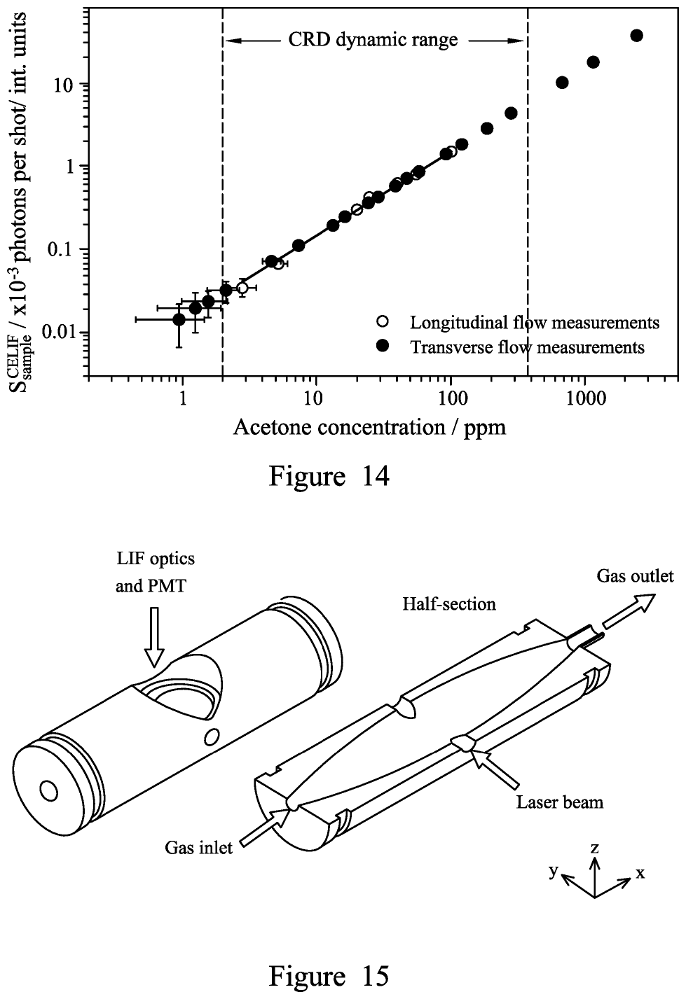

FIG. 14 shows each CELIF signal from the transverse flow of the samples converted into acetone concentration using the calibration factor.

FIG. 15 shows a first flow body of the present invention, flow body 1.

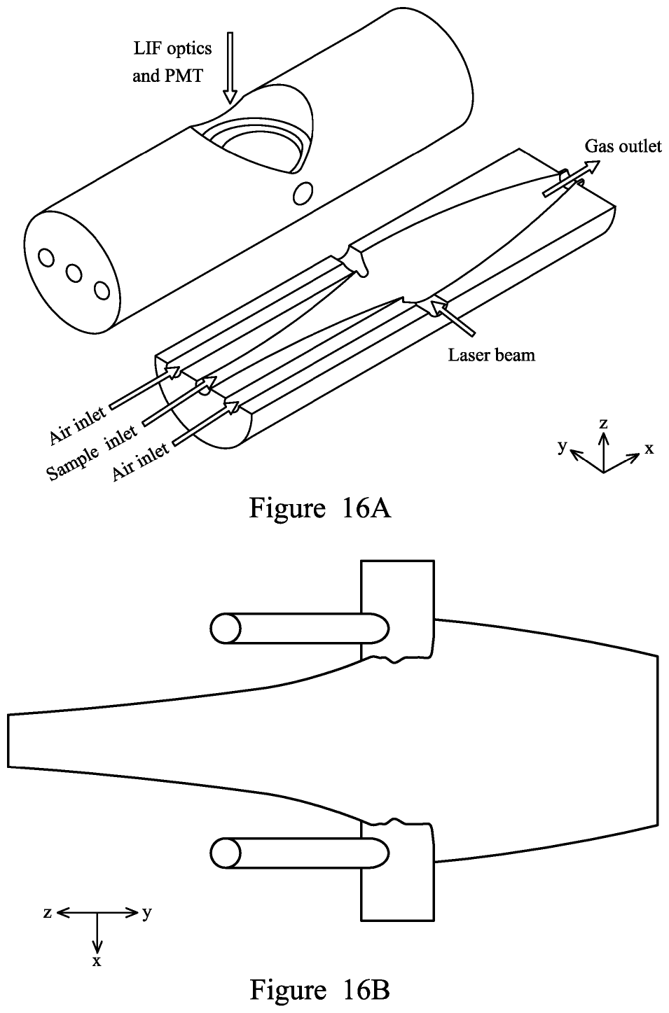

FIGS. 16A and 16B shows a second flow body of the present invention, flow body 2.

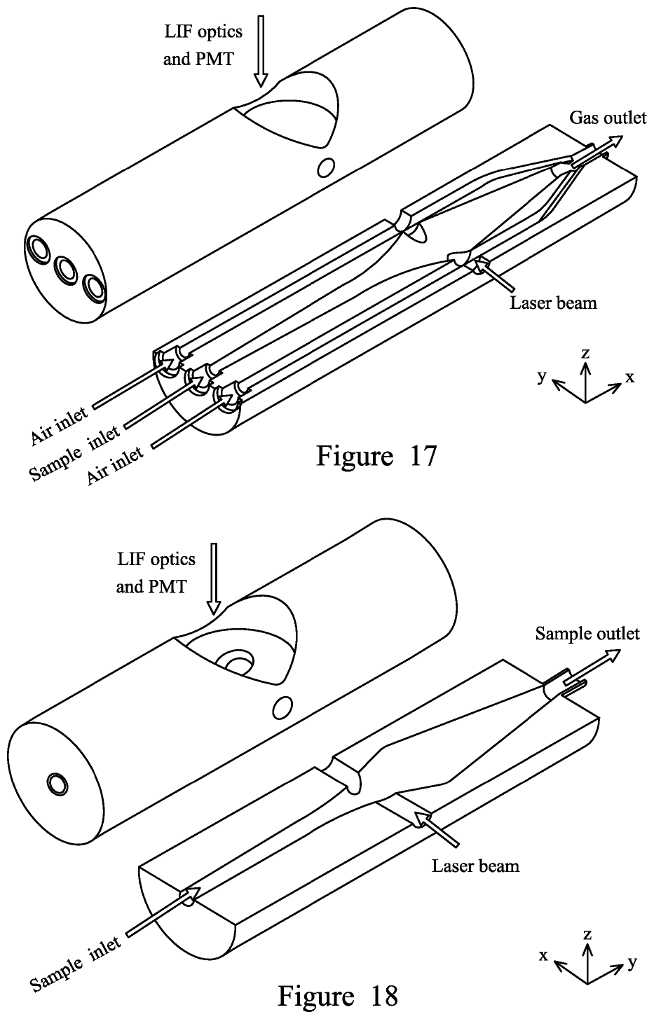

FIG. 17 shows a third flow body of the present invention, flow body 3.

FIG. 18 shows a fourth flow body of the present invention, flow body 4.

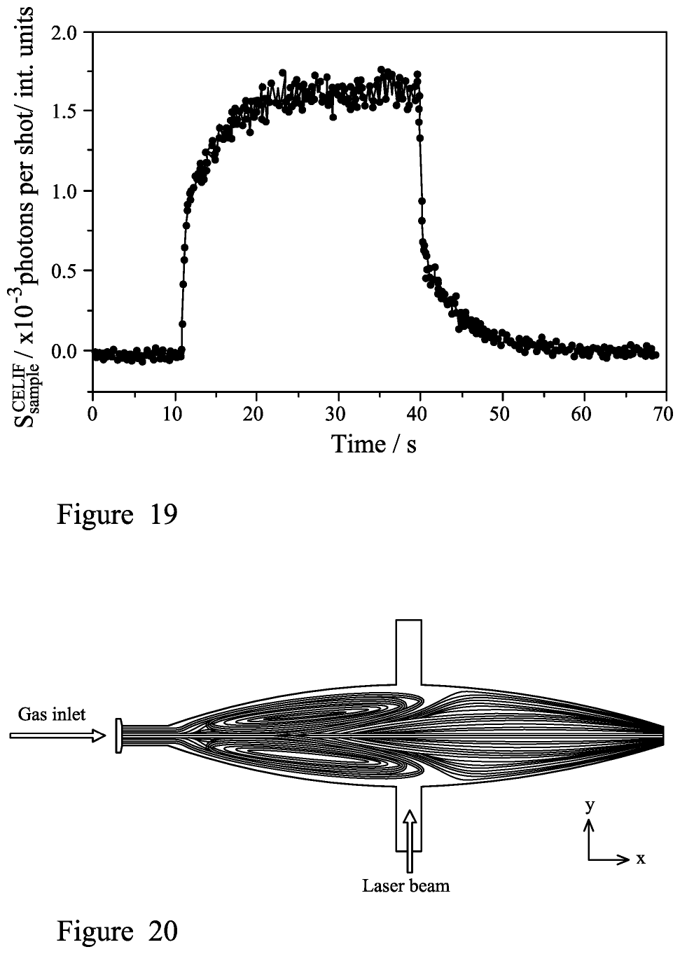

FIG. 19 shows a CELIF measurement taken on the first flow body shown in FIG. 15.

FIG. 20 shows the path of the flow traces calculated in computational fluid dynamics simulation of flow body 1.

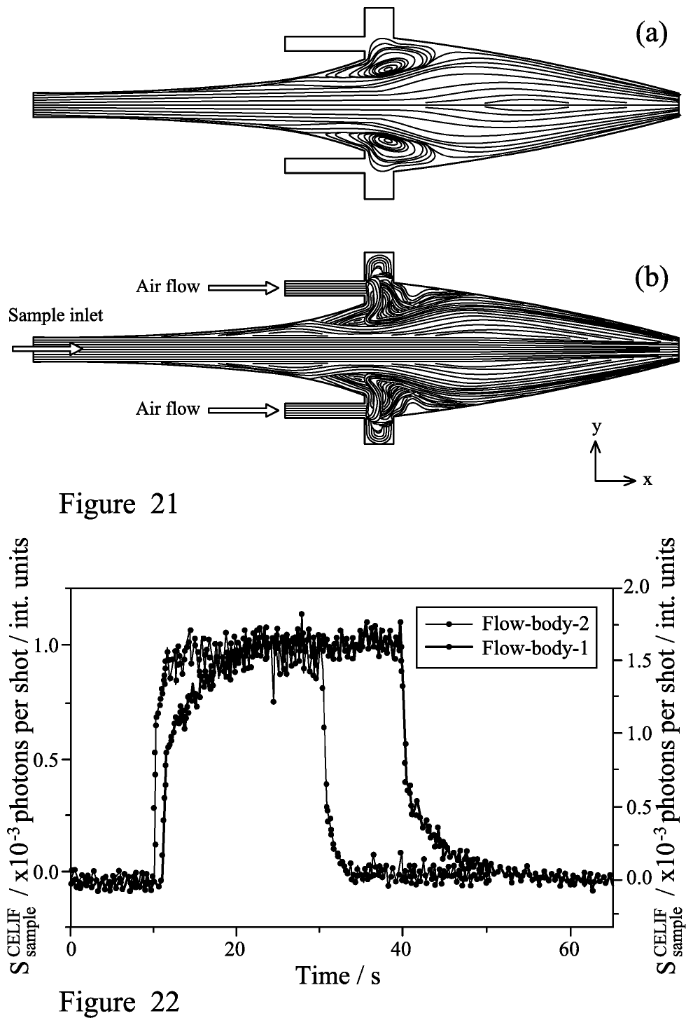

FIG. 21 shows a trace profile of a computational fluid dynamics simulation of flow body 2 with the side flow channels not in use (FIG. 21(a)) and in use (FIG. 21(b)).

FIG. 22 shows a CELIF signal from flow body 1 compared to flow body 2.

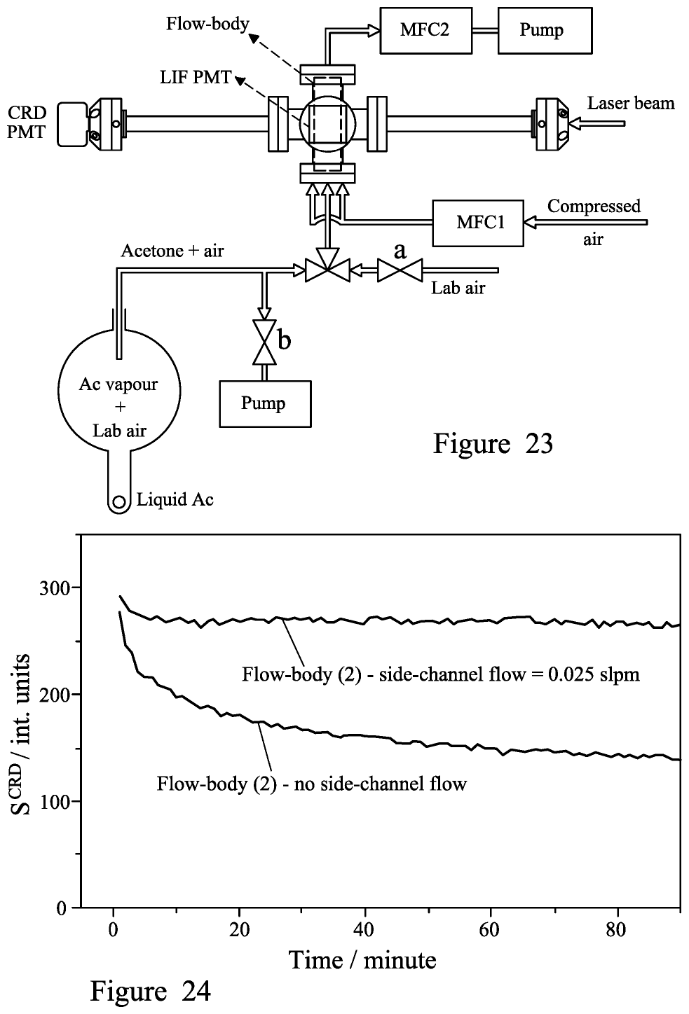

FIG. 23 shows a modified sampling apparatus to accommodate operation of the side flow channels.

FIG. 24 shows a CRD signal recorded on flow body 2 with and without side flow channels.

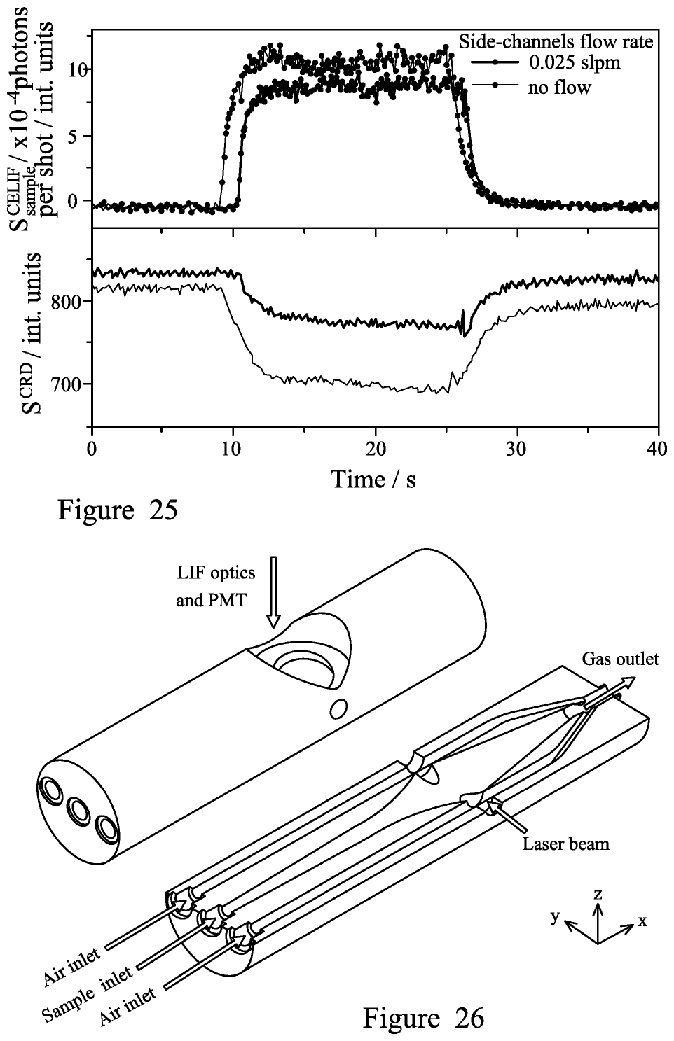

FIG. 25 shows a plot of two CELIF response time measurements, one with side channel flow, and the other one with no side channel flow.

FIG. 26 shows a flow body of the present invention, flow body 3, also represented in FIG. 17.

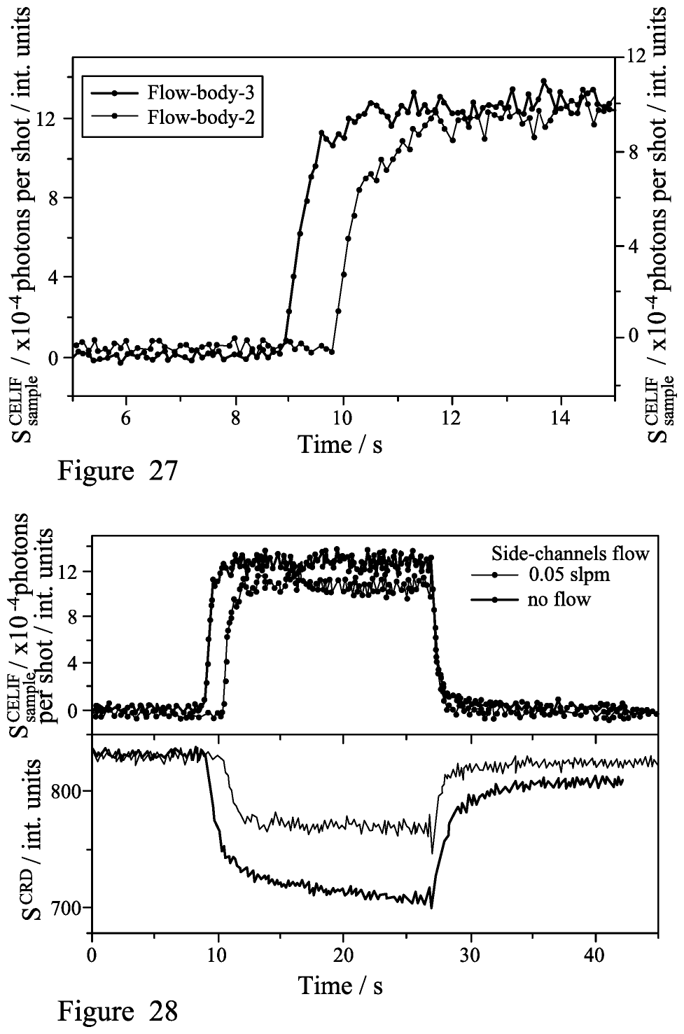

FIG. 27 shows a CELIF response time test made on flow body 3 using the sampling method presented in FIG. 23 made first with no side channel flows compared with the similar measurement made with flow body 2.

FIG. 28 shows two CELIF response time measurements made with flow body 3 with the first measurement made with no side channel flows and the second one made with 0.05 slpm air flow through each channel.

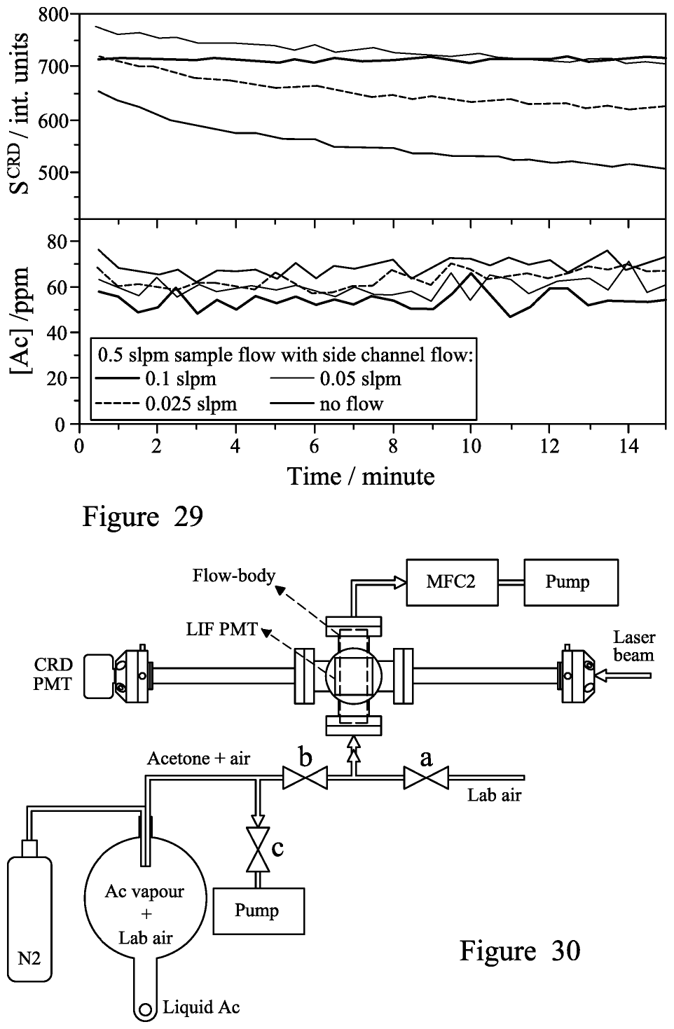

FIG. 29 shows time lapse acetone CELIF measurements made with flow body 3 at different side channel flow rates.

FIG. 30 shows a gas sampling system which does not involve using a 3-way valve and interrupting the pressure inside the cavity.

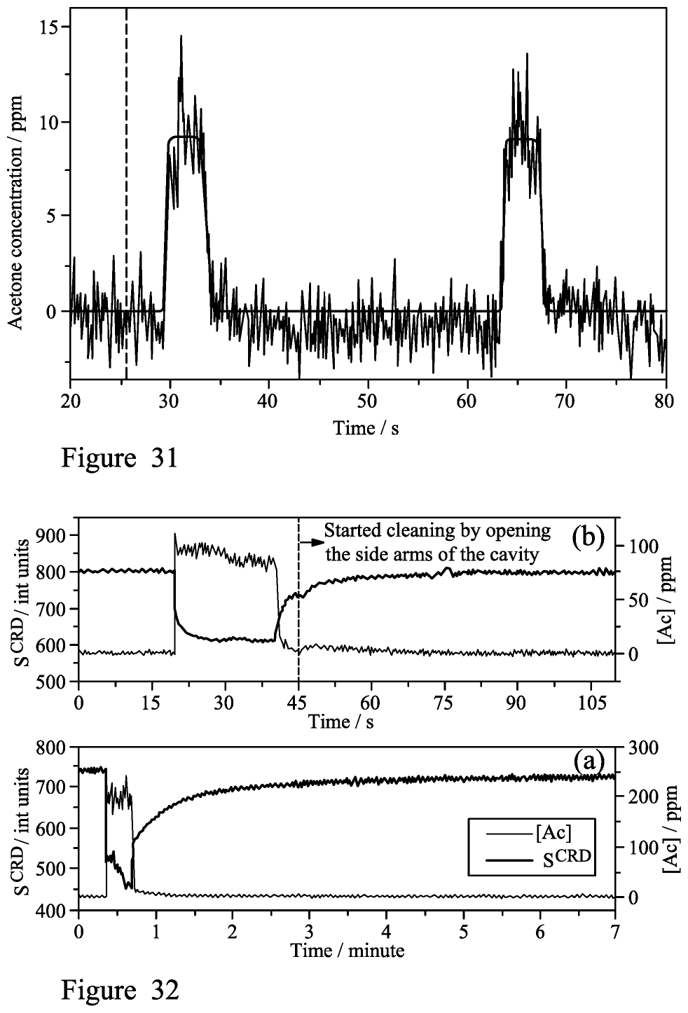

FIG. 31 shows a CELIF response time measurement made on the sampling system of FIG. 30.

FIG. 32(a) shows a CELIF measurement made by allowing lab air to flow into flow body 3 without side channel flows, flowing an acetone-air mixture for about 20 s, then turning the flow back to only lab air.

FIG. 32 (b) shows a second CELIF measurement under the same conditions as FIG. 32(a) except this time the air inlets in the side arms of the cavity, near the cavity mirrors, were open after the acetone CELIF measurement was made to allow air to flow through the system from 3 inlets.

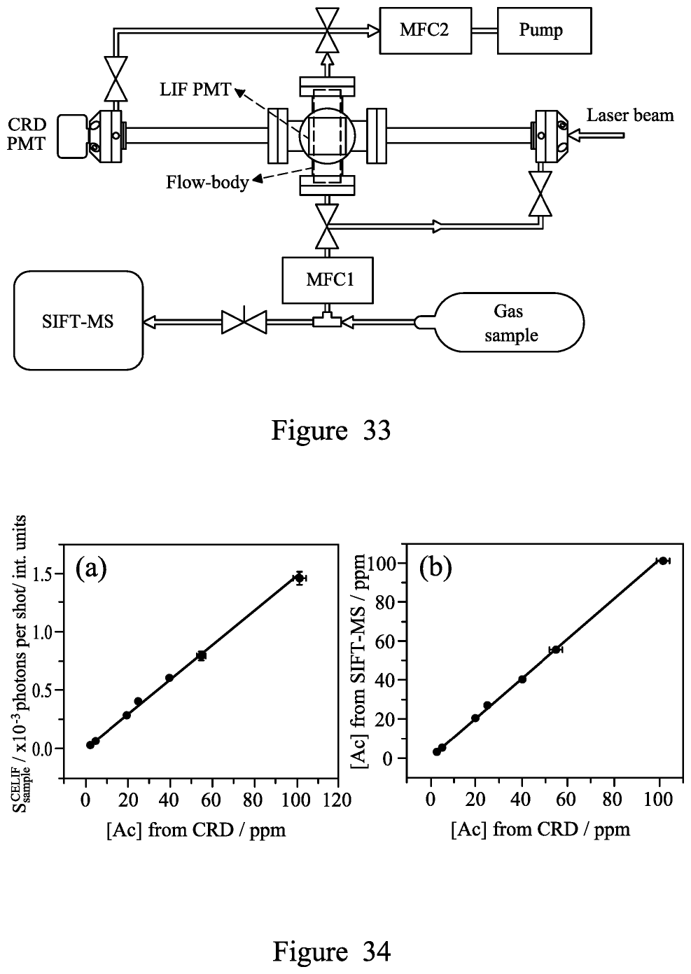

FIG. 33 shows a gas sampling setup including a SIFT-MS instrument for use in the CELIF validation measurements.

FIG. 34 shows a comparison between CRD and SIFT-MS measurements of acetone concentration.

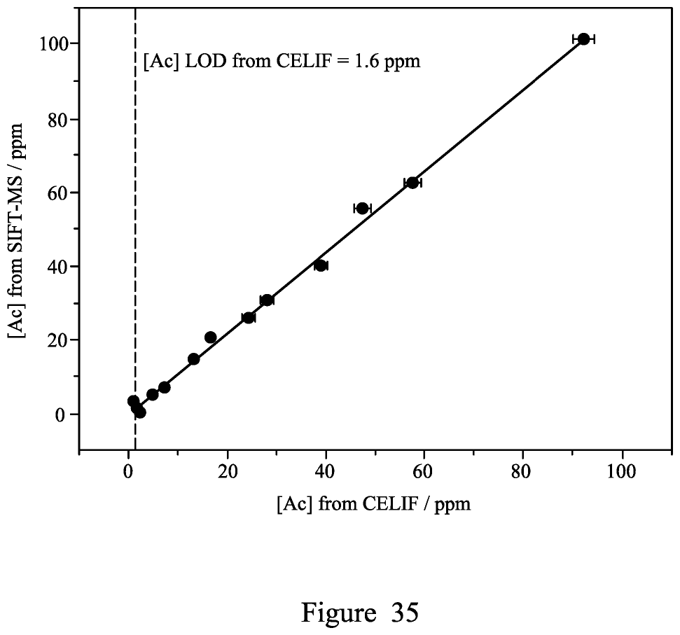

FIG. 35 shows CELIF validation measurements using both the standard gas mixture and the home-made mixtures.

FIG. 1 shows the geometry of an embodiment where the laser/cavity axis intersects the sample flow at right angles. As can be seen from FIG. 1, the sample flow has been denoted as the x axis. The direction of the laser and the optical cavity, being formed from the pair of reflective mirrors located on an optical axis, has been denoted as the y axis. The z axis refers to the axis of the fluorescence detector.

The width of the chamber or dimension of the chamber along the y axis and in the vicinity of the laser beam may have a maximum value so that a maximum amount of the gaseous sample is irradiated by the laser beam. In alternative embodiments, the width of the chamber or the internal dimension may reach a maximum value after or before the sample flow passes the laser beam path.

Expanding the chamber in the dimension of the laser/cavity axis (the y axis) and the fluorescence detection axis (z axis), compared to the chamber inlet and the chamber outlet, reduces the relative area of the two holes for the laser compared to the internal surface area of the chamber. If the internal surfaces of the chamber are designed well, then the flow will be parallel across the laser beam inlet and the laser beam outlet minimising loss of sample gas into the side arms that enclose the cavity.

The laser beam inlet and the laser beam outlet are preferably not sealed by windows. This is because windows could absorb and/or scatter light from the laser beam reducing the effectiveness of the ring-down aspect of the technique.

As shown in FIG. 2, with further examples in FIGS. 5A and 5B, the intersection of the laser by the sample flow may be at any point along the laser beam within the cavity. The axis of the fluorescence detection optics intersects at the same point as the laser and sample flow. However, the intersection of the axis does not need to be at right angles. This is shown in FIGS. 3 and 4. In a preferred embodiment the x axis, y axis and z axis are at right angles to each other, as shown in FIG. 1.

In an embodiment, the apparatus may further comprise a second fluorescence detector located at a location not on the cavity axis and arranged to provide a first signal in response to fluorescence within the cavity. Such an embodiment is depicted in FIG. 6.

Configuration of the Chamber

The chamber within the flow body controls the flow of sample across the laser beam path. Preferably, the chamber is configured such that the sample flow is laminar across the laser beam path with limited eddies or recirculation or capture of the sample within the beam path.

As discussed above, the width of a cross section of the chamber taken parallel to the sample flow (in the x-z plane and/or the x-y plane, referring to FIG. 1) may increase from the inlet to a maximum value in the vicinity of the laser beam path and may decrease towards the outlet, so that the velocity of the sample gas decreases as it flows towards the vicinity of the laser beam and increases from the vicinity of the laser beam to the outlet.

FIGS. 7 to 10, 15, 16A, 17, 18, 20, 21 and 26 show exemplary shapes of the chamber.

In embodiments, the chamber may follow the general configuration of the chamber discussed above, whereby the cross section of the chamber taken parallel to the sample flow (in the x-z plane and/or the x-y plane, referring to FIG. 1) may increase from the inlet to a maximum value in the vicinity of the laser beam path and may decrease towards the outlet, with the width being maintained at a maximum for a distance along the length of the chamber. Such a configuration is shown in FIG. 9 with a cross section in the x-z plane. This has the advantage of providing an increased signal to the photon detector, the flow of sample gas through the beam being laminar preventing repeat passage of the analyte molecules which have already been irradiated and which may be bleached by exposure to the laser beam which in turn could be detrimental to the fall time.

In an embodiment, the width of the chamber (in the x-z plane and/or the x-y plane, referring to FIG. 1) may reach a maximum at or downstream of the laser beam path in order to optimise laminar flow across the laser beam.

The chamber may comprise three sections: a first section adjacent to the sample inlet; a second, central section; and a third section adjacent to the sample outlet. The second section may be absent, accordingly, in embodiments the chamber is formed of the first section and the third section.

In an embodiment the cross-sectional configuration of the chamber in the y-z plane (the plane parallel to the beam path and parallel to the axis of the fluorescence detector) is generally circular at the inlet. Accordingly, the first section may have a circular cross section. In an embodiment the circular cross section of the first section of the chamber increases in size in the direction of the sample flow. The second section may similarly have a circular cross section. However, the circular cross section of the second section remains a constant size. The third section also may have a circular cross section with the size of the cross section decreasing in the direction of sample flow until the third section meets the sample outlet.

In an embodiment the cross-sectional configuration of the chamber transitions from a circular configuration to a configuration where the cross section is circular with flattened sides. The point at which the circular cross section transitions into the flattened circular cross section marks the transition from the first section to the second section.

A flattened circular cross sectional configuration may take the form of a circle where a pair of opposing flat sides are present or where two pairs of opposing flat sides are present. Accordingly, the second section may have such a cross-sectional configuration. The cross-sectional configuration may transition from circular to quadrilateral, via the flattened circular shape previously mentioned. Such a transition may occur within the second section. Continuing in the direction of flow towards the outlet, the cross-sectional configuration may transition from a quadrilateral to a flattened circular shape to a circular shape or from a flattened circular shape to a circular shape, as appropriate, with the size of the cross section decreasing towards the outlet. The transition from a quadrilateral to a flattened circular shape to a circular shape or from a flattened circular shape to a circular shape marks the transition from the second section to the third section.

The discussion above revolved around a cross section of the chamber in the y-z plane (the plane parallel to the beam path and parallel to the axis of the fluorescence detector. We now consider the chamber cross section in the x-y plane (parallel to the laser beam path, parallel to the laser beam path, and perpendicular to the axis of the fluorescence detector).

The first section may be configured as an expanding cone starting from the narrowest point adjacent to the inlet and expanding to a widest point in the direction of the sample flow. The widest point of the cone is where the first section and second section meet or where the first section and the third section meet. The second section may be configures as a cylinder. The third section may be configured as a narrowing cone going from a widest point adjacent to the second section or the first section where the second section is absent and decreasing in width to a narrowest point of the cone adjacent to the sample outlet. The second section and the third section may meet at the widest point of the third section.

The first section may have a cross section in the x-y plane and/or the x-z plane shaped as an expanding cone starting from the narrowest point adjacent to the inlet and expanding to a widest point in the direction of the sample flow. The widest point of the cone is where the first section and second section meet or where the first section and the third section meet (in embodiments where there is not a second section). The second section may have a cross section in the x-y plane and/or the x-z plane shaped as quadrilateral shape or a quadrilateral shape with rounded corners. The third section may have a cross section in the x-y plane and/or the x-z plane shaped as a narrowing cone going from a widest point adjacent to the second section or the first section where the second section is absent and decreasing in width to a narrowest point of the cone adjacent to the sample outlet. The second section and the third section may meet at the widest point of the third section.

The cross-sectional shape of the chamber may have a plane of symmetry. The cross sectional shape of the chamber may have one or two planes of symmetry.

The second section may be the laser beam inlet and the laser beam outlet or may comprise the laser beam inlet and the laser beam outlet. The laser beam inlet and the laser beam outlet are on opposite sides of the second section and are positioned to provide a path for the laser beam through the chamber. The laser beam inlet and the laser beam outlet may alternatively be positioned in the first section or in the third section. Optionally, the laser beam outlet and the laser beam inlet are present at wider areas of the first or third section.

In an embodiment the chamber comprises a first section having a cross section parallel to the laser beam path and perpendicular to the axis of the fluorescence detector shaped as an expanding cone starting from the narrowest point adjacent to the inlet and expanding to a widest point and a third section having a cross section parallel to the laser beam path and perpendicular to the axis of the fluorescence detector shaped as a narrowing cone going from a widest point and decreasing in width to a narrowest point of the cone adjacent to the sample outlet, wherein the first section and the third section meet at their respect widest point.

It would be evident from the discussion above that the chamber may have an internal shape that is symmetrical. The plane of symmetry may be present at the laser beam path.

In embodiments the flow body further comprises one or more side flow channels. Preferably, the flow body comprises two side flow channels. The side flow channels may be placed at any position within the flow body yet outside of the chamber. In embodiments, two side flow channels flank the chamber running parallel to the sample flow direction. The side flow channels have an inlet to the chamber to allow gas to flow from the side flow channels into the chamber.

The side flow channels may be configured to provide air or inert gas inlet channels which may be provided at or upstream adjacent the laser beam inlet and outlet apertures in the flow body, so that air or inert gas flows across the laser beam inlet or outlet preventing ingress of sample, analyte-containing gas into the laser beam inlet or outlet apertures. This may reduce or avoid sample, analyte-containing gas becoming trapped in the inlet or outlet apertures and consequently being measured multiple times or concentrating in the cavity.

In embodiments the air or inert gas channels (which may be referred to as side flow channels) may join the chamber at the same point as the laser beam inlet and laser beam outlet. Alternatively, the air or inert gas channels may join the chamber proximate to the sample outlet.

In embodiments the side flow channels extend along the length of the flow body from the sample inlet end of the flow body to the sample outlet end of the flow body.

In embodiments the side flow channels may intersect with the optical cavity such that gas flows across the laser beam path to reduce or prevent ingress of sample, analyte-containing gas into the optical cavity. In addition, or in alternative embodiments, the side flow channels communicate with the chamber proximate the sample outlet via an inlet.

We have discovered that non-laminar flow of the analyte gas may give a lag in the response of the detector, particularly if repeated sampling of several breaths takes place with a resultant blurring of the observed output signal.

Further, transverse flow of the sample gas through the flow body may give a higher limit of detection of the CELIF device, of more than 2000 ppm, superior to the cavity ring down measurement and covering the dynamic range of the CELIF device.

In previously used methods for analysis of acetone in breath, a patient may exhale into a bag. This may provide a sort of average value but gives an artificial result.

Further, the procedure needs to be repeated whenever a measurement is required.

The present invention allows a patient to breathe naturally with a measurement being provided in real time for each exhaled breath. This facilitates measurements of acetone levels in breath of children or sleeping or unconscious patients. Also, measurements of animals' breath is facilitated.

The flow body of the present invention allows CELIF measurements of flowing samples. The flow body was designed so that it delivers the sample from the about 6 mm (about ¼ inch) tubing system before the laser beam path, to the measurement region with gradual increase in diameter, which may match the width of the field of view of the fluorescent detector. Then, gradually decrease the width of the flow body to match the 6 mm (about ¼ inch) tubing system at the sample outlet. As seen in FIG. 11 the flow body was made of a 125 mm long aluminium cylinder with an outer diameter of 33.8 mm. In the middle section there were two 6 mm holes opened to the flow body, the laser beam inlet and the laser beam outlet, which were required to allow the laser beam to pass and interact with the sample, whilst minimising the mixing between breath samples and the stagnant air in the cavity. As seen in the half-section view of the flow body in FIG. 11, when viewed from the top (the x-y plane), the flow body concavely expands from a narrow dimension (optionally 4 mm) to a wider dimension (optionally 25 mm) the laser beam interacts with the sample, in a sample width within the field of view of the LIF PMT, and then returns to the initial diameter at the gas outlet.

When viewed from the side (in the x-z plane), the sample inlet concavely expands from a narrow dimension to a wider dimension (however, this is less than the dimension that the flow body expands to when viewed from the top the x-y plane). The wider dimension in this case may be 8 mm). The flow body then returns to the initial diameter at the sample outlet.

At the top of the flow body there is an aperture (optionally in the size of 25 mm) for the LIF optics to be fitted.

The flow body is placed inside a six-way cross. The six-way cross consists of the laser beam path, the fluorescence detector and the sample flow. FIG. 1 represents this six-way cross in the absence of any components of the device. FIG. 11 demonstrates the 6-way cross with a schematic view of the device including components. With the fluorescence detection optics fitted in the flow body and the six-way cross, the LIF PMT extends out from a top flange of the six-way cross (not shown in FIG. 11). The flow of the gas samples was created by attaching the inlet of the flow body with a first mass flow controller (Alicat, MC-5SLPM) (MFC 1) that controls the pressure of the sample inlet and keeps it at 1 bar. The sample outlet of the flow body is connected to a second mass flow controller (MFC 2) that controls the mass flow rate of the flowing sample. The upstream of MFC 1 was connected to the pressurised samples, and the downstream of MFC 2 was connected to a rotary pump.

Before testing the transverse flow measurements through the flow body it was essential to first compare CELIF measurements of static gas samples which fill the whole volume of the cavity with CELIF measurements of flowing gas samples through the cavity axis, which fill the same volume, to test whether the flow of gas affects the concentration measurements.

These tests showed that static CELIF measurements do not reflect the real acetone concentration in a sample. This may be due to optical bleaching of the stagnant acetone molecules in the cavity, which does not occur while flowing the sample as the molecules that interact with the laser get replaced quickly preventing bleaching. Thus, CRD and CELIF measurements must be made with flowing gas samples.

It was found that for the real-time CELIF measurements to be quick and to minimize the residence time of the samples in the CELIF device, gas samples should flow transversely through the flow body, from the sample inlet to the sample outlet at an angle to the beam path (optionally perpendicular) and not along the beam path, this can be seen in FIG. 1.

Signals of different standard acetone-nitrogen gas mixtures were measured once with longitudinal flow and once with transverse flow, with a flow rate of 0.5 slpm in each flow direction. The measurements of each gas mixture were compared and are shown in FIG. 12. The CELIF signals from transverse flow were plotted together with the CELIF signals from longitudinal flow of the same gas. A linear fit with a slope of 1.02±0.03 proves the agreements between transverse and longitudinal flow CELIF measurements.

FIG. 13 shows a comparison of the LIF and CRD signals between transverse and longitudinal flow of the same gas samples. Surprisingly, it was observed that transverse flow from the sample inlet to the sample outlet (as per the present invention) preserves the laser intensity at the LIF probe region compared to longitudinal flow along the length of the beam (not forming part of the present invention). Without being bound by theory it is believed that this beneficial effect of the mode of the present invention is due to the absence of absorption in side arms of the flow body which form the laser beam inlet and the laser beam outlet. As such, the LIF signal for the same gas sample is strengthened. It also strengthens the CRD signal which makes it possible to measure the time integrated CRD signals at high acetone concentrations. This makes flow of the sample gas from the sample inlet to the sample outlet (transverse flow) ideal to extend the dynamic range of CELIF measurements.

CELIF calibration followed by CELIF measurements of different samples were made to explore the lower and higher limits of detection, dynamic range, of the CELIF device. Standard acetone-nitrogen gas mixtures of acetone concentration in the range between 2-100 ppm were used to do the CELIF calibration. The outlet of the standard gas bottles was connected to the mass flow controller that controls the pressure at the cavity inlet. The outlet of the CELIF device was connected to the second mass flow controller that controls the flow rate of the gas through the cavity.

Each CELIF calibration measurement was made by first measuring the laser independent background, γ, by blocking the laser then recording the LIF signal. Then measuring the background CELIF signal for an acetone-free sample, which was measured by longitudinally flowing 1 bar of N₂ at 0.5 slpm. Then, the sample CELIF signal was measured by longitudinally flowing some of the acetone-nitrogen gas mixture at a pressure of 1 bar and a flow rate of 0.5 slpm. The sample CELIF measurement was recorded after 1 minute of the gas flow to ensure that the cavity pressure which was controlled by the mass flow controller was stable. Each background and sample CELIF measurement was an average of 1500 laser shots. The cavity was flushed with flowing N₂ after each measurement. After each measurement the cavity is flushed with N₂, for 2 minutes along the sample path, followed by 2 minutes along the laser path and then 1 minute along the sample path. A typical calibration measurement is based on five individual measurements of analyte concentrations ranging from 0 ppm to 100 ppm. Next, to produce a calibration curve the CELIF signals were plotted against the acetone concentration as measured by CRD. Then, a straight line with zero intercept was fitted to the data, yielding a calibration factor [symbol] ′=1.49±0.03×10⁻⁵. The three standard deviation (3σ) limit of detection (LOD) of the cavity ring-down measurements was calculated to be 2 parts per million (ppm), when averaging over every 1500 laser shots in 100 ms, using

$${ρ}_{LOD}^{3 σ} = \frac{3 \sqrt{2} δ {τ}_{0}}{c σ {τ}_{0}^{2}}$$

where, τ₀ is the analyte-free ring-down time, δτ₀ is the error in τ₀ and σ is the absorption cross section of acetone at 266 nm. The higher limit of detection of the CRD measurements was estimated from the ability to fit a CRD transient to a single exponential decay to measure the ring-down time, which gave acetone concentration of 390 ppm as the higher limit.

Next, a high concentration acetone-nitrogen mixture was prepared in a 0.5 L gas cylinder by first pumping down the cylinder, then releasing some of the gas acetone into it, then topping up the cylinder with 9 bar of gas N₂. The mixture then was left to mix thoroughly, for example for about 1 hour with the help of the heated side arms of the mixing cylinder. The gas line was connected to the mass flow controller and the flow body inlet, and the flow body exit was connected to the second mass flow controller. Then, γ was measured by blocking the laser and recording the LIF signal, and the background CELIF signal was measured by transversely flowing 1 bar of N₂ through the flow-body at 0.5 slpm. After that, 1 bar of the acetone-nitrogen mixture was released into the chamber of the flow body with a flow rate of 0.5 slpm, and after 1 minute of the gas flow a CELIF measurement was recorded which was an average of 1500 laser shots. Next, the cavity was flushed with N₂, and the rest of the gas mixture was diluted by topping it up with N₂ and was left to mix for about 15 minutes. Then, another set of background and sample CELIF measurements were taken as above. The procedure was repeated many times until the limit of detection was reached.

FIG. 14 shows each CELIF signal from the transverse flow of the samples converted into acetone concentration using the calibration factor, [symbol] ′, and was appended to the CELIF calibration graph. The acetone concentration CELIF limit of detection was calculated to be 1.6 ppm using

$${C}_{LOD}^{3 σ} = \frac{3}{{𝒦}^{′} {S}_{0}^{CRD}} \sqrt{\frac{2 {S}_{bg}^{LIF}}{n}}$$

FIG. 14 also shows that even at acetone concentrations as high as 2000 ppm, the concentration was measurable. Thus, the transverse flow of the gas through the flow body gives an upper limit of detection of the CELIF device of more than 2000 ppm, winning over the cavity ring-down measurement and covering the required dynamic range for the CELIF device.

For the apparatus of the present invention to be clinically accepted, the acetone CELIF measurement must be able to follow the real-time breathing pattern, by responding quickly to any change in the acetone concentration. The CELIF signal must rise and reach the maximum acetone concentration in less than 1 s, then fall and return to the baseline level before the next breath arrives. The rise time of the measurement is defined as the time taken for the signal to rise from 10% to 90% of the steady state signal, and the fall time of the measurement is the time taken for the signal to fall from 90% to 10% of the steady state of the signal.

### EXAMPLES

Four flow bodies forming part of the invention were tested. Flow body 1 is shown in FIG. 15. Flow body 2 is shown in FIG. 16. Flow body 3 is shown in FIG. 17. Flow body 4 is shown in FIG. 18.

### Example 1

The performance of flow body 1, shown in FIG. 15, was tested using a mixture of acetone in air. The gas sampling setup was changed to be able to create a continuous stream of gas mixture at atmospheric pressure, without the need for premixing or worrying about controlling the pressure of the flow body inlet. A mixture of acetone vapour and air was created by putting a few drops of liquid acetone in a 3 L glass bulb which was open to the atmosphere. A sampling tube was inserted into the glass bulb, while the other end of the tube was connected to a T-piece. One side of the T-piece was connected to a mass flow controller (MFC 1), which was connected to a pump, to give a continuous flow of 5 slpm through the sampling tube, and prevent accumulation of acetone vapour in the tube. The other side of the T-piece was connected to a 3-way valve, which allowed either lab air or a gas sample to flow through the flow body, with a flow rate of 0.5 slpm controlled by the mass flow controller at the exit of the flow body (MFC 2).

CELIF Response Time with Flow Body 1

A CELIF background measurement was made by measuring the laser independent background, γ, by blocking the laser then recording the LIF signal. Then measuring the background CELIF signal by flowing lab air through the flow body. Next, the CELIF signal was recorded every 100 ms by averaging 1500 laser shots, while the 3-way valve was set to flow lab air to record a baseline for the measurement, then after a few seconds, the 3-way valve was turned to the sample side and allowing the sample to flow for a few seconds before turning the 3-way valve back to the lab air side. The CELIF measurement was stopped when the baseline returned to the initial value.

The result of this measurement is shown in FIG. 19. The baseline from 0-12 s indicates air flow, while the rise in the CELIF signal followed by the plateau indicates the acetone-air sample flow. Then the fall of the signal at 40 s and the baseline after that indicates the switching of the 3-way valve to the lab air side. The steady state of the acetone CELIF signal confirms that the gas sampling method discussed above provides constant concentration of acetone in air.

The rise time of the CELIF signal was 6.2±0.2 s and the fall time was 6.6±0.2 s. Therefore, at least 12 s was needed for a single acetone measurement to be made using this flow body.

Sample Diffusion Through Side Arms with Flow Body 1

Time lapse acetone CELIF measurements were made to test whether the gas samples flowing thought the flow body diffuse into the side arms of the cavity, or if the samples being diluted by the stagnant air in the cavity side arms. A mixture of acetone in air was made using the method discussed above (creating a mixture of acetone vapour and air by putting a few drops of liquid acetone in a 3 L glass bulb which was open to the atmosphere). The mixture was flowed through the flow body at flow rate of 0.5 slpm for more than 1 hour, while the LabView program was recording the CRD, LIF and CELIF signals every 1 minute, averaging 1500 laser shots for each measurement.

The results showed that both the time-integrated CRD signal and the LIF signal decayed with time. As the gas extended into the side arms through the two laser holes in the flow body, the sample length increased and the light intensity at the middle section of the cavity decreased, which caused both the CRD and LIF signals to decay with time. Even though the sample diffused into the side arms of the cavity, the CELIF signal was constant for the entire period of the experiment as expected.

Computational Fluid Dynamics Simulations

To investigate the reason behind the long rise and fall time of the CELIF measurement made using flow body 1, and to find a suitable solution to this issue, Computational Fluid Dynamics (CFD) simulations were performed to visualise the theoretical flow through the flow body. Autodesk CFD was used for this purpose. These CFD simulations of the different designs of the flow body were made by J. Landes, “Optimisation of Analyte-Gas Flow Through a CELIF Sensor for Breath Acetone”, Masters thesis, Durham University, 2018, the contents of which is incorporated herein by reference.

Torun a CFD simulation for a flow body, a CAD drawing of the object must be created and imported into the CFD software. The materials of the various parts of the CAD object must be assigned including the flow volume. The boundary conditions of the flow system must be set including the volume flow rates of the sample gas at the inlets and the static pressure on the outlets. The samples used in the CELIF experiment contained only a small concentration of acetone, which would have a negligible impact on the flow of the gas. Thus, the fluid material “air” was used in all of the simulations. The following step was to create a mesh for the system for the 3D simulation. The mesh is where the simulation is actually performed, which is a series of tetrahedral elements which approximate the geometry of the CAD object. The partial differential equations used by the CFD software to describe the flow of fluid do not have solutions for complex systems, therefore the system is divided into smaller subsystems within which analytical solutions can be found. A fine mesh will match the geometry and result in an accurate simulation, however this costs a large amount of computer memory and long computational time. A coarse mesh will result in a fast simulation, but will produce inaccurate results.

The general method used to create a mesh for an object in the CFD software for the simulations performed in this project is as follows: first, using the auto-size function a simple coarse mesh was created. Then, the size adjustment was used to refine the mesh, which increases or decreases the size of the mesh elements by up to a factor of 5. Using this, the mesh was refined to give an element count between one and two million. Next, “surface refinement” and “gap refinement” were enabled to refine the mesh on the surfaces and between gaps in the CAD geometry. The final step was to adjust the wall layer settings. A wall layer is a mesh along the wall of the flow volume used to simulate the boundary layer. Therefore, it is important that the wall layer is thick enough and contains enough layers to resolve the whole boundary layer. The number of layers within the wall layer settings was set to 9, the layer factor, which determines the layer thickness, was set to 0.2 and the layer gradation, which controls the rate of growth of the wall layers, was kept at the automatic setting. This method generated the initial mesh for each CFD simulation.

Certain control settings had to be chosen to run the CFD simulations, such as if the flow is laminar or turbulent and if the fluid is incompressible. A flow is characterized to be laminer (smooth) or turbulent (rough) by a dimensionless parameter called Reynolds number: Re=(ρdυ)/η where ρ and η are the fluid's density and viscosity, dis the diameter of the flow channel, and υ is the flow velocity.

Values of Re<2000 predict a laminar flow, whereas values of Re>2500 usually indicate that flow will be turbulent. In a laminar flow system, some vortices, or localized swirling, can separate from the central streamlines within a flow channel, which unlike turbulent flow, fluid in vortices is composed of slowly moving currents and streamlines. The Mach number M is a dimensionless quantity representing the ratio of flow velocity to the speed of sound. At values of M<1, the compressibility of the fluid can be ignored.

The values of the Mach and Reynolds numbers of our flow system are significantly lower than the Mach and Reynolds number at which a fluid beings to show compressibility and turbulence, respectively. Therefore, it was determined that the simulations will use the approximations that the flow is laminar and incompressible to reduce the computational cost.

The simulation results must be mesh independent. Thus, mesh adaptation was enabled to ensure this was true for the simulations. This is a program which analyses the results and refines the mesh where needed. The results after running mesh adaptation were always mesh independent.

CFD Simulation of Flow Body 1

A CFD simulation was performed on flow body 1. The CAD geometry used for this simulation was as shown in FIG. 15, except the holes for the LIF optics were removed as only the basic shape of the flow body was being studied, and closed arms were added to simulate the optical cavity. The simulation was performed as described in the previous section. The boundary conditions for the flow were a volume flow rate of 0.5 L/min placed on the inlet, and a static gauge pressure of 0 Pa placed on the outlet.

A set of traces spread evenly over the inlet were plotted to see the path the gas takes as it enters the flow body. The traces are a packet of data points which enter the system and map out the path the gas takes based on the velocity of the fluid.

The path of the traces of flow body 1 are shown in FIG. 20. The traces show that large vortices formed either side of the main flow. The vortices formed here due to the sharp increase in the width of the flow body after the inlet, resulting in boundary layer separation. No vortices formed in the perpendicular plane as the width increases much more gently. The boundary layer split the sample flow into three distinct regions: the two vortices and the main flow tube, where the flow is laminar, and is shaped by both the walls of the flow body and the vortices. The widest section of the flow body chamber was positioned at about 3 cm to the right of the middle of the flow body. Within the vortices, the traces had an average residence time of 24.6±0.9 s, with the residence time defined as the time a trace stays in the system before exiting through an outlet. This is about twice as much as the measured rise and fall time of the CELIF measurement made with flow body 1. This is likely to be caused by the simplification of the design made for the simulations. The long residence time of the traces in the vortices is most likely a cause of long rise and fall times of the acetone CELIF measurement. Therefore, removing vortices present within the fluid flow path could improve the CELIF measurement and the measurement speed should be increased. Accordingly, it is an aim of certain embodiments of the present invention to avoid the formation of vortices within the chamber of the flow body.

### Example 2

Flow body 2 was designed with a chamber similar to the shape of the laminar flow found in the simulation of flow body 1. The CAD drawing for this new design is shown in FIG. 16A. In the x-y plane, there is a smooth widening of the flow body, with a convex shape, in order to prevent boundary layer separation. In the z-x plane, flow body 2 was kept unchanged from flow body 1 as no vortices were found in this plane. There is a smooth concave increase in the flow width, with the widest point lying in the middle of the flow body. This flow body includes two side flow channels to be used to flow air to remove any sample that would get trapped in a vortex in order to maintain a smooth flow profile, and to prevent the sample from diffusing into the side arms of the cavity.

Adding these two channels required the modification of the main flow tube body found in the simulation of flow body 1. This included increasing the width of the chamber after the laser beam path. The side channels were designed so that the air flow in the side channels would join the sample flow next to the beam inlet and beam outlet. This is so that at the point of measurement, minimal mixing would occur, reducing any effect the side channel flow would have on the sample concentration. This flow body prototype was 3D printed from a plastic material (PLA polylactic acid) after being studied with CFD simulations.

CFD Simulation of Flow Body 2

The design of flow body 2 shown in FIG. 16A could not be meshed with the CFD software because of the complex design of the side channels. Thus, for the CFD simulation only, the geometry of flow body 2 was simplified as shown in FIG. 16B. This included using cylindrical side channels and increasing the gap between the side channels and the main flow at the point they meet. To fit the side channels into the system, their diameter had to be reduced to 3 mm and the width of the flow body after the cavity had to be increased.

Similar CFD simulation to that of flow body 1 was performed on the simplified flow body 2. First, only the main flow tube was being studied, so no volume flow rate boundary condition was placed on the side channel inlets. The trace profile mapping the flow through flow body 2 is shown in FIG. 21 (a). It shows that small vortices form in the edges of the flow at the middle of the flow body. This is likely to be caused by the modification of the design and by increasing the width of the chamber after the cavity. However, the absence of a steep increase in the width after the side channels, should result in smaller vortices.

Next, the effect of air flow through the side channels was studied using CFD simulations. The magnitude of the side channel flow rate was chosen so that the average velocity of the sample at the middle of the flow matches the velocity of the air in the side channels. From the previous simulations it was found that the averaged velocity of the sample at a sample flow rate of 0.5 L/min was 3 cm/s. Using this and the cross section area of the side channel, the flow rate at the inlet of each side channel was set to 0.013 L/min. The trace profile mapping the flow through the main flow chamber and the side channels of flow body 2 is shown in FIG. 21 (b). It shows that the side channel flow allows the sample to flow smoothly through the system by preventing the formation of vortices in the sample flow by filling the region where boundary layer separation would occur. This should correspond to a faster rise and fall times of CELIF signals. Because the width of the sample flow was reduced by the side channel flows, the width of the LIF optics hole in flow body 2 was reduced to 16 mm (compared to 25 mm in flow body 1). This is to reduce the sight of view of the LIF PMT so that only fluorescence from the acetone molecules can be detected.

### Performance of Flow Body 2

CELIF Response Time with Flow Body 2

A CELIF response time test was made using flow body 2, first without flowing air through the side channels, and using the same experiment and sampling method as Example 1 as what was used with flow body 1. The inlets of the side channels were covered to prevent any flow through them. The CELIF signal from this measurement was plotted together with the CELIF signal measured with flow body 1 as shown in FIG. 22. The rise and fall times of the CELIF signal when measured with flow body 2 were 1.5±0.1 s and 1.6±0.2 s respectively, which is, despite not flowing any air through the side channels, a significant improvement from Example 1, flow body 1.

The Effect of the Side Channel Flows

In order to test the effect of the side channel flows, the sampling method presented in Example 1 had to be slightly modified. As shown in FIG. 23, (MFC 1) was attached to the inlet of the side flow channels of flow body 2 and was set to flow air at a flow rate of 0.05 slpm (0.025 slpm through each channel). The continuous sample gas flow from the glass bulb was maintained by connecting the sampling tube to a shut off valve, valve (b), followed by the pump. Valve (b) was partially open to set the flow rate to about 5 slpm. It was found that turning the 3-way valve from air to sample and vice versa, causes a quick pressure drop in the cavity as the middle of the 3-way valve is a dead volume. This causes a delay of a few seconds for the cavity to recover the atmospheric pressure. Thus, another shut off valve, valve (a), was added to the lab air side and was slightly restricting the air flow to aid in maintaining stable pressure when turning the 3-way valve. The flow body exitmass flow controller (MFC 2) was set to flow the gas at flow rate of 0.55 slpm, where the main 0.5 slpm is for the sample flow, and 0.05 slpm is for the side channel flows.

Time lapse acetone CELIF measurements were made to test whether the gas samples flowing through flow body 2 diffuse into the side arms of the cavity. A mixture of acetone in air was made and was flowed through the flow body at a flow rate of 0.5 slpm, first with no side channel flows, for more than 1 hour, while the CRD, LIF and CELIF signals were recorded every 1 minute, averaging 1500 laser shots for each measurement. This measurement was repeated with flowing air through the side channels at a flow rate of 0.025 slpm through each channel.

The results are shown in FIG. 24. Similar to what happened with flow body 1, when there were no side flow channels, the CRD signal quickly decayed as the gas extended into the side arms through the two laser holes in the flow body. However, when flowing the sample along with flowing air through the side channels the CRD signal decayed for only the first 10 s of the sample flow then maintained a smooth flow with constant CRD signal. This suggests that the side channel flow helps in constraining the sample in the middle of the flow body but not completely preventing the diffusion into the side arms. Faster flow rate through the side channels caused mixing of the air with the sample and reducing the sample concentration.

Next, the effect of the side channel flow on the CELIF measurement was tested.

Using the sampling method shown in FIG. 23, two CELIF response time measurements were made, one with side channel flow, and the other one with no side channel flows. Both measurements were made using the same gas mixture and were made successively. These two measurements are plotted together as shown in FIG. 25. While the side channel flow did not improve the rise and fall time of the CELIF measurement, it clearly caused a reduction in the acetone concentration as measured by the CELIF signal. This is likely to be caused by how the side channel flows joins the sample flow and then mixing with and diluting the sample.

Furthermore, the CRD signal of the measurement with side channel flows recovers faster than the CRD signal of the measurement without side channel flows when switching the 3-way valve to air after flowing the acetone mixture. This further reinforces the conclusion that the side channel flows helps in reducing the amount of the sample gas diffusing into the laser beam inlet and outlet of the chamber, and agrees with the results of the diffusion test. Also shown in the CRD signals in FIG. 25, at about t=26 s the effect of the 3-way valve switching on the pressure of the cavity can be seen. This should be taken into account when measuring the response time of the CELIF instrument.

The speed of the CELIF measurement with flow body 2 showed significant improvement compared to flow body 1. Even though the side channel flow proved to reduce the amount of the sample diffusing into the side arms, it caused reduction of the sample concentration. Taking these issues into account, a third flow body was designed as discussed in the following section.

### Example 3

The vortex-free flow shape found in the CFD simulations of flow body 1 was modified in flow body 2 to fit in the side flow channels, which caused the formation of vortices in the sample flow in flow body 2. The design of the new flow body (flow body 3), is shown in FIG. 26. This new flow body was designed from the exact vortices free flow shape found in FIG. 20. To prevent the sample flow from diffusing into the side arms of the cavity, the flow body may have side channels to create a barrier between the main sample flow and the cavity arms. The side flow channels were designed to run parallel to the main sample flow and join the sample just before the gas outlet to reduce the chances of mixing between the air and the sample at the measurement region.

CFD Simulation of Flow Body-3

CFD simulation similar to the previous simulations was run for this flow body. The flow rate boundary condition for the sample inlet was set to 0.5 L/min, and the side channel flow rate was determined using the same method as flow body 2 and was set to 0.02 L/min through each channel. The boundary condition for the gas outlet was set to 0 Pa gauge pressure. The traces showed that no vortices are formed in the sample flow, and that no mixing occurs between the sample flow and the side channels flow at the middle of the flow body. The traces show that the sample would fill the total width of the main flow volume, thus, when this flow body was 3D printed from PLA plastic the LIF optics hole was set to be 19 mm.

Performance of Flow Body-3

CELIF Response Time with Flow Body-3

Similar to previous flow body designs, a CELIF response time test was made using the sampling method presented in FIG. 23. The measurement was made first with no side channel flows, and was compared with the similar measurement made with flow body 2, as shown in FIG. 27. The rise and fall times of the CELIF measurement taken with flow body-3 with no side channel flows were 0.8±0.1 s and 1±0.2 s respectively. This is faster than the CELIF measurement made by flow body 2.

The Effect of the Side Channel Flows

Two CELIF response time measurements were made with flow body 3 using the same gas mixture. The first measurement was made with no side channel flows, and the second one was made with 0.05 slpm air flow through each channel. The two measurements are plotted together in FIG. 28. Despite the simulations showing that no mixing occurs between the air in the side channels and the sample, the experiment shows that the side channel flows dilute the acetone concentration in the sample.

However, beneficially the slope in the CRD signal for the measurement without side channel flows, compared to the flat CRD signal in the measurement with side channel flows suggests that the side channel flows act as barriers between the flow regions and reduce the amount of the sample gas that diffuse into the side arms.

Time lapse acetone CELIF measurements were made with flow body 3 at different side channel flow rates. A mixture of acetone in air was made and was flowed through the flow body at a flow rate of 0.5 slpm for 15 minutes, while the CRD, LIF and CELIF signals were recorded every 1 minute, averaging 1500 laser shots for each measurement. This measurement was repeated flowing air through the side channels at flow rates of 0.025, 0.05 and 0.1 slpm through each channel.

The results are shown in FIG. 29. Similar to what happened with previous flow-bodies, when there was no side channel flows, the CRD signal quickly decayed as the gas extended into the side arms through the two laser holes in the flow body. However, when flowing the sample along with flowing air through the side channels the rate of the decay decreased with increasing side channels flow rate. Side channel flow rate of 0.1 slpm through each channel was able to prevent the diffusion of the sample into the side arms,

However, as shown in the acetone concentration plot, flowing air through the side channels with even a small flow rate caused dilution of the sample. Even with the improved design of the side flow channels, mixing between the sample and the side channel flows occurred, thus, the use of side channel flows should be avoided.

### Example 4

CELIF Response Time

At this stage, no trials were made on patients. Thus, a gas sampling method which does not involve using a 3-way valve and interrupting the pressure inside the cavity was implemented. FIG. 30 shows the sampling method. Similar to the previous methods, a few drops of acetone were put in a 3 L glass bulb, which was open to the atmosphere. A nitrogen gas was supplied continuously into the glass to dilute the acetone-air mixture. A sampling tube was inserted into the glass bulb, and the other end of the tube was connected to a T-piece. One end of the T-piece was connected to the pump through a shut off valve, valve c, and the other end of the T-piece was connected to another shut off valve, valve b. Valve c was partially open to allow a continuous stream of sample gas with a flow rate of about 5 slpm, to prevent the acetone vapour from accumulating inside the tube. Valve b is connected through another T-piece to a third shut off valve, valve a, which was always open to lab air. The other end of the T-piece is connected through a short tube to the sample inlet of flow body 3 (Example 3). The inlets of the side channels in the flow body were blocked. While valve a was always open, a sample could be flowed through the flow body by opening valve b. by doing this the pressure inside the cavity remained undisturbed.

The CELIF response time measurement was made by first allowing the lab air only to flow through the system, then valve b was open for a few seconds and then quickly closed to allow the sample to join the air flow into the system. This was repeated a second time, while the CRD, LIF and CELIF signals were recorded every 100 ms, averaging 1500 laser shots in each measurement.

The result is shown in FIG. 31. The real time acetone concentration is shown, as measured by CELIF. The straight line superimposed over the real time acetone measurement is a fit of the data of the form:

$$y = \frac{{A}_{1}}{( 1 + exp ( - \frac{t - {t}_{r 1}}{{τ}_{r 1}} ) ) \times ( 1 + exp ( \frac{t - {t}_{f 1}}{{τ}_{f 1}} ) )} + \frac{{A}_{2}}{( 1 + exp ( - \frac{t - {t}_{r 2}}{{τ}_{r 2}} ) ) \times ( 1 + exp ( \frac{t - {t}_{f 2}}{{τ}_{f 2}} ) )}$$

where, for the first acetone peak, A₁ is the acetone concentration, tri is the centre of the rising edge, τᵣ₁ is the time constant of the rising edge, t_{f1} is the centre of the falling edge and τ_{f1} is the time constant of the falling edge. The rest of the constants are the same for the second acetone peak. From that the 10% rise time

of the signal peak, t₁₀, is

$$\frac{1}{1 + exp ( - \frac{{t}_{10}}{{τ}_{r}} )} = \frac{1}{10} ,$$

$$exp ( - \frac{{t}_{10}}{{τ}_{r}} ) = 9 ,$$

$${t}_{10} = - {τ}_{r} ln ( 9 ) .$$

And the 90% rise time of the signal peak, t₉₀, is

$$\frac{1}{1 + exp ( - \frac{{t}_{90}}{{τ}_{r}} )} = \frac{9}{10} ,$$

$$exp ( - \frac{{t}_{90}}{{τ}_{r}} ) = \frac{1}{9} ,$$

$${t}_{90} = - {τ}_{r} ln ( 9 ) .$$

Thus, the 10-90% rise time of the signal is

2τᵣ ln(9)

and the same follows for the 90-10% fall time of the signal,

2τ_{f} ln(9)

From the fit it was found that the 10-90% rise time of the CELIF measurement was 370±15 ms, and the 90-10% fall time was 850±21 ms, where the errors are from one standard deviation. The rise and fall time of the CELIF measurement are fast enough to follow a real breath pattern. While the two times should be equal, the longer fall time is likely to be caused by some acetone sticking into the tube fittings before the flow body which takes some time to clear up.

### Example 5

### Cavity Cleaning Between CELIF Measurements

To test how long it takes the cavity to return to CELIF background level after introducing a sample of high acetone concentration, a CELIF measurement was made by allowing lab air to flow into flow body 3 without side channel flows, flowing an acetone-air mixture for about 20 s, then turning the flow back to only lab air, as shown in FIG. 32 (a). The CRD signal shows that there was acetone build up in the side arms. The slow (several minutes) decay of the signals back to the baseline level after flowing a sample of acetone was probably caused by the fact that a high acetone concentration has been flowing through the flow body for about 20 s. This decay is not seen in FIG. 31 where the “breaths” are much shorter (<5 s) and more representative of a real breath.

Next, the same measurement was repeated as shown in FIG. 32 (b), but this time the air inlets in the side arms of the cavity, near the cavity mirrors, were open after the acetone CELIF measurement was made to allow air to flow through the system from 3 inlets: the flow body inlet and from both side arms of the cavity and exit the system through the flow body outlet, flushing any acetone residuals that were stuck on the cavity walls. By doing this, the signals decayed back to the background level within only 30 s, presenting a quick method to clean the cavity and removing any acetone from the system after breath measurements.

While monitoring real patients breath, with a lower acetone concentration than the acetone used in this example, we could tolerate some acetone build up in the side arms as long as it does not affect the CELIF measurement. However, eventually the build up will become intolerable, and the cavity will need to be flushed by the method described above. The time after which the cavity will need to be cleaned depends on the amount of acetone concentration being monitored. Therefore, it is recommended to clean the cavity at least every 1 minute while monitoring a real patient's breath with acetone concentration of more than 30 ppm, and every 2 minutes if the acetone concentration is less than that.

### Example 6

Flow Body 4

The design of the flow body is shown in FIG. 18. The flow body was made to be fully fitted inside the 6-way cross, thus, the total length was 125 mm and the outer diameter was 33.8 mm. The laser apertures were positioned upstream of the widest part of the flow body internal volume. This minimised the flow and diffusion of sample gas through the laser apertures into the cavity sidearms as confirmed by computational fluid dynamics simulations. The diameter of the LIF optics hole was made only 10 mm to fit the width of the sample. The sample inlet and outlet were ¼ inch to fit with the other tubing system of the experiment. The flow body was machined from aluminum. The flow body was painted inside and out with an optical absorber coating (Alion MH2200) which was proved to reduce the amount of the LIF background level by absorbing the scattered light inside the system.

FIG. 31 shows the measurement (blue line) of a rapidly varying acetone concentration at a flow rate of 0.5 slpm and a fit (red line) according to the equation

$$[ Ac ] ( t ) = {\sum }_{i = 1}^{N} \frac{{A}_{i}}{( 1 + exp ( - \frac{t - {t}_{r , i}}{{τ}_{r , i}} ) ) \times ( 1 + exp ( \frac{t - {t}_{f , i}}{{τ}_{f , i}} ) )}$$

in which [Ac](t) is the measured acetone concentration as a function of time, t, Aᵢ is the amplitude of one acetone pulse, t_{r,i} and t_{f,i} are the times of the rising and falling edges, respectively, and τ_{r,i} and τ_{f,i} are the rise and fall times, respectively. From the fit it was found that the 10-90% rise time of the CELIF measurement was 370±15 ms, and the 90-10% fall time was 850±21 ms, where the errors are one standard deviation.

In order to investigate the accuracy of the CELIF instrument and to fulfill the requirement for the CELIF instrument to be medically approved, the concentrations of acetone measured by the CELIF instrument must be compared with a proven analytical technique. A selected ion flow tube mass spectrometer (SIFT-MS) instrument (Voice200Ultra—Syft Technologies) was provided by Anatune to the Department of Chemistry at Durham University and was used to validate the CELIF instrument.

SIFT-MS is a new analytical technique for the simultaneous, real-time quantification of several trace compounds in air or breath samples as described in P. Španiěl and D. Smith, “Progress in SIFT-MS: Breath analysis and other applications,” Mass Spectrometry Reviews, vol. 30, pp. 236-267, July 2010; D. Smith and P. Španiěl, “SIFT-MS and FA-MS methods for ambient gas phase analysis: Developments and applications in the UK,” Analyst, vol. 140, no. 8, pp. 2573-2591, 2015; and D. Smith and P. Španiěl, “Selected ion flow tube mass spectrometry (SIFT-MS) for on-line trace gas analysis,” Mass Spectrometry Reviews, vol. 24, no. 5, pp. 661-700, 2005, the contents of which are hereby incorporated by reference.

CELIF Validation Measurements

A SIFT-MS instrument (Voice200Ultra—Syft Technologies) was provided by Anatune and was used for the CELIF validation measurements. It was placed such that concomitant readings could be taken on the same gas sample. The gas sampling setup is shown in FIG. 33. A T-piece was added after a gas cylinder outlet to split the gas sample between the CELIF and the SIFT-MS instruments to allow simultaneous measurements by both instruments. The gas flow path in the CELIF device was made such that it can be easily switched between the longitudinal CRD measurements along the cavity axis and the transverse CELIF measurements through the flow body. A needle valve was used to restrict the flow to the SIFT-MS instrument.

The CELIF device was first calibrated by using standard acetone-nitrogen gas mixtures in the range of 1-100 ppm (SIP Analytical, SIPCYL 110 LTR non-refillable can filled certified grade acetone in nitrogen). Each gas mixture was measured simultaneously by the two instruments. CRD and LIF measurements were recorded for CELIF calibration by flowing the gas longitudinally through the cavity axis and simultaneously the acetone concentration in the sample was measured by the SIFT-MS. Both the CELIF cavity and the SIFT-MS were flushed between the measurements. Each CELIF measurement was an average of 1500 laser shots and was recorded using a 0.5 slpm sample gas flow. Each SIFT-MS measurement was an average of acetone concentrations measured over 20 s of gas flow, with a measurement interval of 140 ms.

TABLE 1
A summary of the SIFT-MS instrument
(Voice200Ultra - Syft Technologies)
method used for the validation of the CELIF device.

| Carrier gast type | Helium | |
|---|---|---|
| Reagent ion | NO⁺ | (30 amu) |
| Production ion | NO⁺CH₃COCH₃ | (88 amu) |

| | |
|---|---|
| Carrier Flow (tls) | 5.0057 |
| Sample Flow (tls) | 0.3 |
| Tube Temp (C.) | 123.75 |
| Tube Pressure (Torr) | 0.6348 |
| Reaction Time (ms) | 8.203 |

Next, to validate the CELIF measurements with the SIFT-MS measurements, the procedure above was repeated using the same standard gas bottles but flowing the gas samples transversely through the flow body and using the previous CELIF calibration to calculate the acetone concentration. More validation measurements were also made using a homemade acetone-nitrogen mixture and a series of dilutions where each concentration was measured by CELIF and the SIFT-MS simultaneously. The SIFT-MS instrument was not optimised to measure acetone concentrations above about 100 ppm, thus the validation measurements were restricted to acetone concentrations between 1 ppm (CELIF limit of detection)-100 ppm.

The used SIFT-MS method is summarised in table 1. Surprisingly, a SIFT-MS instrument dependent correction factor was needed to correct for the acetone reaction rates which was important for accurate quantification of our validation measurements. In the (Anatune) lab, a gas sample that should give 6 ppm acetone was generated and measured with SIFT-MS using all the possible reagent ions, and from the data generated the reaction rates were amended. This yielded that a correction factor of 1.519 must be multiplied by the acquired SIFT-MS validation data.

The CELIF calibration measurements and the comparison between CRD and SIFT-MS measurements of acetone concentration are presented in FIGS. 34 (a) and (b) respectively. The slope 1.02±0.02 in the fitting equation suggests that the obtained acetone concentrations using both methods are consistent. The CELIF validation measurements using both the standard gas mixture and the home-made mixtures are shown in FIG. 35. The acetone concentration simultaneous measurements with the CELIF device and the SIFT-MS instrument agree with each other with a linear fitting slope of 1.1±0.1. This SIFT-MS validation test proves that the acetone concentration measurements by the CELIF device are reliable and fast and can be used for measurements of breath samples.

The acetone CELIF measurement procedure, the flow body design and the response time of the CELIF measurements were optimised. We achieved an acetone concentration dynamic range between 1.6-2000 ppm, covering the range of breath acetone concentration a DKA patient might have. The CELIF device was validated with the SIFT-MS.

1. An apparatus for quantifying an analyte in a gas or liquid phase sample comprising:
a light source with an emission spectrum that overlaps with an absorption of the analyte, a pair of reflective mirrors located on an optical axis to form an optical cavity, the cavity having a sample inlet and a sample outlet;
a fluorescence detector located at a location not on the cavity axis and arranged to provide a first signal in response to fluorescence within the cavity;
a photon detector located axially external to the cavity and arranged to provide a second signal;
wherein the apparatus including the light source, the cavity and the axial photon detector comprises a cavity-enhanced absorption spectrometer;
wherein the apparatus including the fluorescence detector is configured to comprise a cavity-enhanced laser-induced fluorescence (CELIF) spectrophotometer;
means for supplying an analyte-free gas sample or an analyte-containing gas sample to the cavity through the inlet;
a processor adapted to receive a first signal from the fluorescence detector and a second signal from the axial photon detector, and further adapted to provide a measurement of the analyte concentration in the sample:
wherein a flow body is located between the sample inlet and sample outlet, the flow body comprising a chamber extending along a direction of sample flow between the sample inlet and the sample outlet, the flow body further comprising a light source inlet and a light source outlet outlet arranged to provide a path for a light source through the chamber, the path extending transversely of the direction of sample flow;
the flow body further comprising an aperture communicating with the fluorescence photon detector in a direction transverse to the path of the light source.

2. The apparatus of claim 1, wherein the light source is a laser and the path of the light source is a laser beam.

3. The apparatus of claim 2, wherein the laser is a sub-nanosecond-pulsed Nd:YAG laser.

4. The apparatus of claim 1, wherein the flow body further comprises one or more flow channels.

5. The apparatus of claim 1, wherein the chamber has a dimension perpendicular to the direction of sample flow, wherein the dimension increases from a minimum value at the sample inlet to a maximum value in the vicinity of the light source path and decreasing to a value smaller than the maximum value at the sample outlet.

6. The apparatus of claim 1, wherein the dimensions of the chamber increase or decrease smoothly.

7. The apparatus of claim 1, wherein the direction of sample flow, the laser beam path and the fluorescence detector aperture are perpendicular or orthogonal and intersect at a single point.

8. The apparatus of claim 1, wherein the surface of the chamber may have a smooth profile configured to minimize turbulent flow of the sample gas, or formation of eddies, during passage through the chamber.

9. The apparatus of claim 1, wherein the width of a cross section of the chamber taken parallel to the sample flow increases gradually from the inlet to a maximum value in the vicinity of the laser beam path (for example just before the laser beam path) and may decrease towards the outlet, so that the velocity of the sample gas decreases as it flows towards the vicinity of the laser beam and increases from the vicinity of the laser beam to the outlet

10. The apparatus of claim 1, wherein the chamber may comprise three sections: a first section adjacent to the sample inlet; an optional second, central section; and a third section adjacent to the sample outlet.

11. The apparatus of claim 10, wherein the first section has a circular cross section and the circular cross section of the first section increases in size in the direction of the sample flow.

12. The apparatus of claim 11, wherein the second section has a circular cross section, remaining at a constant size in the direction of sample flow.

13. The apparatus of claim 12, wherein the third section has a circular cross section and the circular cross section decreases in size in the direction of sample flow.

14. The apparatus of claim 13, wherein the first section is configured as an expanding cone starting from the narrowest point adjacent to the inlet and expanding to a widest point in the direction of the sample flow.

15. The apparatus of claim 12, wherein the second section is configured as a cylinder.

16. The apparatus of claim 13, wherein he third section is configured as a narrowing cone going from a widest point adjacent to the second section or the first section and decreasing in width to a narrowest point of the cone adjacent to the sample outlet.

17. A method of quantifying an analyte in a gas phase sample comprising the steps of: providing apparatus in accordance with the first aspect of the invention; introducing an analyte-free gas sample into the chamber obtaining a background signal; introducing a flowing reference sample containing a quantity of the analyte into the chamber and obtaining a reference signal;
processing the background signal and reference signal to obtain an absolute absorption coefficient of the reference sample and to provide a calibration signal of the CELIF spectrophotometer;
introducing a specimen gas sample containing the analyte into the chamber obtaining a specimen signal;
processing the specimen signal and the calibration signal to obtain a measurement of the absolute concentration of the analyte in the specimen sample;
wherein the measurement is made while the specimen sample is flowing through the chamber.

18. A method as claimed in claim 17, wherein the analyte concentration is measured repeatedly overtime intervals of 100 ns to 5 ms.

19. A method of claim 17, wherein an average value for the analyte concentration is measured over a period from 1.0 ms to 0.03 s.

20. A method of detecting or monitoring Type 1 diabetes in a patient comprising the step of measuring acetone in exhaled breath using the method as claimed in claim 17.

21. A method of detecting ketone acidosis in a patient comprising the step of:
measuring acetone in exhaled breath using apparatus as claimed in claim 1.

22. A method as claimed in claim 21, wherein the apparatus further comprises a breath collector connected to an inlet of the apparatus.

23. A method as claimed in claim 22 wherein the breath collector comprises a manifold, face mask or breathing tube.
